(19) United States
(12) Patent Application Publication
(10) Pub. No.: US 2024/0391963 A1
(43) Pub. Date: Nov. 28, 2024

(54) CONTROL OF SUBUNIT STOICHIOMETRY IN SINGLE-CHAIN MSP NANOPORES

(71) Applicant: THE UAB RESEARCH FOUNDATION, Birmingham, AL (US)

(72) Inventors: Michael Niederweis, Birmingham, AL (US); Mikhail Pavlenok, Birmingham, AL (US)

(73) Assignee: THE UAB RESEARCH FOUNDATION, Birmingham, AL (US)

(21) Appl. No.: 18/693,641

(22) PCT Filed: Sep. 23, 2022
(86) PCT No.: PCT/US2022/044550

§ 371 Date: Mar. 20, 2024

Related U.S. Application Data
(60) Provisional application No. 63/247,872, filed on Sep. 24, 2021.

(51) Int. Cl. C07K 14/35; C07K 1/113; C12P 21/02; C12R 1/19; G01N 33/487

(52) U.S. Cl. CPC C07K 14/35; C07K 1/1136; C12P 21/02; G01N 33/48721; C07K 2319/21; C07K 2319/50; C12R 2001/19

(57) ABSTRACT

A preparing a purified population of single chain Mycobacterium smegmatis porins.

### PRIOR RELATED APPLICATION

This application claims the benefit of and priority to U.S. Provisional Application No. 63/247,872, filed on Sep. 24, 2021, which is hereby incorporated by reference in its entirety.

### STATEMENT REGARDING FEDERALLY FUNDED RESEARCH

This invention was made with government support under grant number R21 HG010543 awarded by the National Institutes of Health. The government has certain rights in the invention.

### BACKGROUND

Nanopore sequencing is often hindered by the very fast translocation of DNA. Control of the DNA translocation rate is currently achieved by DNA-processing enzymes, but this adds complexity and stochastic signals from the motor protein, decreasing the signal-to-noise ratio. In addition, the residual current of single-stranded DNA passing through the pore is determined by four to five nucleotides at each position. These and other limitations result in raw base calling errors of up to 12% for MspA. Therefore, compositions and methods for producing MspAs with improved sequencing capabilites are necessary.

### SUMMARY

Provided herein are single-chain Mycobacterium smegmatis porin (Msps), for example, Mycobacterium smegmatis porin A (MspAs) and methods for preparing a purified population of single chain Msps (e.g., MspAs). The methods comprise (a) expressing in E. coli a polypeptide comprising a single chain MspA, wherein the polypeptide comprises, in the following order, (i) a first affinity tag, wherein the affinity tag is a polyhistidine tag; (ii) a first amino acid linker; (iii) a single chain MspA, wherein the single chain MspA comprises at least a first MspA monomer sequence and a second MspA monomer sequence; wherein the first and second monomer sequence are linked by a second amino acid linker; (iv) a third amino acid linker; and (v) a second affinity tag; (b) recovering inclusion bodies that express the single chain MspAs from the E. coli under denaturing conditions; (c) using Ni-affinity chromatography to obtain one or more fractions comprising single chain MspAs from the inclusion bodies under denaturing conditions; (d) optionally separating the single chain MspAs in the one or more fractions using size exclusion chromatography to obtain a desired fraction comprising MspAs; and (e) purifying the MspAs from the one or more fractions of step (c) or the desired fraction of step (d) using second affinity tag purification under denaturing conditions. Some methods further comprise: (f) refolding the purified MspAs of step (e) in a refolding buffer, wherein the refolding buffer comprises about 50 mM to about 200 mM L-Arginine, about 800 mM to about 1000 mM urea, and 0.5% to about 1.0% OPOE, wherein the buffer has a pH of about 7.0 to about 8.5; and (g) concentrating the refolded MspAs using size exclusion chromatography to obtain a purified population of single chain MspAs.

In some methods, the refolding buffer further comprises about 25 mM to about 50 mM inorganic phosphates (e.g., NaPi) and/or about 150 mM to about 500 mM NaCl. In some methods, the refolding buffer further comprises about 25 mM to about 50 mM inorganic phosphates (e.g., NaPi) and/or about 150 mM to about 300 mM NaCl.

Optionally, the polypeptide comprises (a) a first MspA monomer sequence; (b) a second MspA monomer sequence; and (b) a third, fourth, fifth, sixth, seventh, and eighth MspA monomer sequence or any subset thereof, wherein the first, second, third, fourth, fifth, sixth, seventh and eighth MspA monomer sequence or any subset thereof are arranged consecutively and wherein the second amino acid linker is positioned between any two Msp monomer sequences. In some methods, the second amino acid linker is positioned between every two Msp monomer sequences.

In some methods, the polypeptide further comprises a protease cleavage site positioned between the first amino acid linker and the single-chain MspA and/or a protease cleavage site positioned between the third amino acid linker and the second affinity tag.

The polypeptide optionally comprises one or more first affinity tags, optionally separated by the first amino acid linker. In some methods, the polypeptide comprises one or more second affinity tags, optionally separated by the third amino acid linker. In some methods, the second affinity tag is a streptavidin tag. In some methods, the E. coli is E. coli BL21(DE3)omp8.

In some methods, at least one of MspA monomer sequences is a mutant monomer sequence. In some methods, the mutant monomer sequence comprises a D90N, a D91N, and a D93N mutation. In some methods, the mutant monomer sequence further comprises a D118 mutation, a D134 mutation, and a E139 mutation. In some methods, the mutant monomer sequence further comprises a P97F mutation.

Also provided is a polypeptide comprising a single chain MspA, wherein the polypeptide comprises, in the following order, (i) a first affinity tag, wherein the affinity tag is a polyhistidine tag; (ii) a first amino acid linker; (iii) a single chain MspA, wherein the single chain MspA comprises at least a first MspA monomer sequence and a second MspA monomer sequence; wherein the first and second monomer sequence are linked by a second amino acid linker; (iv) a third amino acid linker; and (v) a second affinity tag.

In some polypeptides, the second amino acid linker is positioned between every two Msp monomer sequences. In some polypeptides, the second amino acid linker is an acidic amino acid linker having a negative charge, for example, a net charge of about −2.0 to about −5.0, at pH 7.0. In some polypeptides, each MspA monomer sequence has at least 95% identity to SEQ ID NO: 1. In some polypeptides at least one MspA monomer sequences is a mutant monomer sequence. In some polypeptides, the mutant monomer sequence comprises a D90N, a D91N, and a D93N mutation. In some polypeptides, the mutant monomer sequence further comprises a D118 mutation, a D134 mutation, and a E139 mutation. In some polypeptides, the mutant monomer sequence further comprises a P97F mutation. In some polypeptides the mutant monomer sequence comprises a D90N, a D91N, a D93N mutation, a P97F mutation, a D118 mutation, a D134 mutation (e.g., a D134R mutation), a E139 mutation (e.g., a E139K mutation). In some polypeptides, the single chain MspA comprises at least three MspA monomers. In some polypeptides, the single chain MspA comprises at least five MspA monomers. In some polypeptides, the single chain MspA comprises at least seven MspA monomers. In some polypeptides, the single chain MspA comprises eight MspA monomers. In some polypeptides, the polypeptide further comprises a protease cleavage site positioned between the first amino acid linker and the single-chain MspA and/or a protease cleavage site positioned between the third amino acid linker and the second affinity tag. In some polypeptides, the polypeptide comprises one or more first affinity tags, optionally separated by the first amino acid linker. In some polypeptides, the polypeptide comprises one or more second affinity tags, optionally separated by the third amino acid linker.

### BRIEF DESCRIPTION OF THE FIGURES

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIGS. 1A-G show the design, production and purification of single-chain MspA. (A) Structure of wt MspA (Protein Database-PDB #: 1UUN) with eight monomers (top view). (B) Model of the covalent peptide linker between two MspA subunits in single-chain MspA (side view). The linker connects the C-terminus of one subunit to the N-terminus of the adjacent subunit. Models in A and B were prepared using Chimera 11. (C) The single-chain mspA gene. Numbered arrows represent translationally fused mspA m2 genes. Boxes with numbers (e.g., L1, L2, L3, etc.) show the linker regions connecting adjacent mspA m2 genes. The scheme is not to scale. (D) Purification of sc₈MspA M2. Samples were loaded onto 10% polyacrylamide gel followed by staining with Coomassie. Lanes: IB—inclusion bodies purified from Omp8 E. coli solubilized in 8 M urea; GE—sample after gel extraction procedure; RF—refolded sample after dialysis. Numbers on the left are molecular weights of the marker in kDa. (E) Denaturation of MspA M2 and ssMspA M2. Coomassie-stained 8% polyacrylamide gel of untreated (−) and samples boiled in 80% (v/v) DMSO (+) to denature proteins. Lanes: M2—octameric MspA M2; sc₈M2—sc₈MspA M2 with eight covalently linked subunits. 1 μg of protein was loaded on each lane. Current trace of sc₈MspA M2 (F) and octameric MspA M2 (G) in a diphytanoylphosphatidylcholine (DPhPC) membrane with an aperture diameter of 1 mm in a Montal-Mueller system. The applied potential was −10 mV, the electrolyte was 1 M KCl, 10 mM Hepes (pH 7.5).

FIGS. 2A-F show channel activity of MspA pores with different subunit stoichiometries. (A). Coomassie-stained 8% polyacrylamide gel with different single-chain MspA PN1 constructs. Lanes: wt—octameric MspA M2; sc-3, -5, -6, -7, -8-single-chain MspA PN1 with three, five, six, seven, eight covalently linked subunits. Octameric MspA M2 and proteins with different subunit composition were boiled in 80% DMSO (+) and compared to untreated protein (−). Note that the 20 kDa band is monomeric subunit of MspA. Equal amounts (2 μg) of each sample were loaded on the gel. (B-F) Current traces of single-channel conductances of scMspA PN1 constructs. Membrane currents were recorded in 1 M KCl, 10 mM HEPES, pH 7.4 electrolyte at −10 mV applied potential. Diameter of the diphytanoyl phosphatidylcholine (DPhPC) bilayer was approximately 1 mm. (B) sc₃MspA PN1: 95 insertion events from 4 membranes; (C) sc₅MsA PN1: 221 insertions from 7 membranes. (D) sc₈MspA PN1: 463 insertion events from 7 different membranes. (E) sc₇MspA PN1: 62 insertions from 5 membranes; (F) sc₈MspA PN1: 150 insertions from 6 membranes.

FIGS. 3A-F show purification and channel activity of single-chain MspA. (A) Scheme of the scMspA gene in the expression vector for protein (sc₈MspA_{dt} M2) purification from E. coli. Eight mspA m2 genes (brown arrow) are flanked by two tags: N-terminal His8-tag and C-terminal Twin-StrepII tag. GS—glycine-serin linker; scMspA—single-chain MspA; StrII—StrepII-tag; TEV—TEV protease recognition site. Not to scale. (B) Coomassie stained 8% polyacrylamide gel with inclusion bodies (IB) from E. coli BL21(DE3) Omp8 and the purified sc₈MspA_{dt} M2 protein. (C) Western blot of the samples shown in B. Proteins were transferred onto PVDF membrane and stained with anti-StrepII-tag HRP-conjugated antibodies. (D) Denaturion of MspA M2 and sc₈MspA_{dt} M2. Coomassie-stained 8% polyacrylamide gel of DMSO treated (+) and untreated (−) samples. Proteins were boiled in 80% (v/v) DMSO to induce denaturing. Note that the 20 kDa band is monomeric subunit of MspA that is visible after DMSO treatment. Lanes: wt—octameric MspA M2; sc₈M2—sc₈MspA_{dt} M2 with eight covalently linked subunits. 3 μg and 1 μg of MspA M2 and sc₈MspA_{dt} M2, respectively, were loaded. (E) Current trace of sc₈MspA_{dt} M2 in 1 M KCl, 10 mM HEPES, pH 7.4 electrolyte at −10 mV applied potential in diphytanoyl phosphatidylcholine (DPhPC) bilayer with an aperture of approximately 1 mm in diameter. The concentration of the protein in the cuvette was 16 ng/ml. (F) Current trace of sc₈MspA_{dt} M2 in 1 M KCl, 10 mM HEPES, pH 7.4 electrolyte at −10 mV applied potential in bi-block polymer (PBD-PEO) bilayer with an aperture of approximately 1 mm in diameter.

FIGS. 4A-G show DNA recognition by single-chain MspA. DNA hairpin translocation experiments with MspA M2 and sc₈MspA_{dt} M2 were performed in free-standing PBD-PEO polymer bilayers supported by wedge-on-pilar 100 μm aperture at 75 mV applied voltage in 1M KCl, 2 M CgmCl, 10 mM Tris, pH 7.5 by patch-clamp amplifier. (A) A free-standing PBD-PEO polymer bilayer membrane is supported by wedge-on-pillar aperture. A single mutant MspA nanopore inserted is shown in the buffer with 2 M GdmCl. A patch-clamp amplifier is connected by Ag/AgCl to measure the ion current through the cis (top) and trans (bottom) chambers. (B) I-V curves of sc₈MspA_{dt} M2 and MspA M2 from −300 to 300 mV with the power spectral density plots at 75 mV shown, in 1 M KCl, 2 M GdmCl, 10 mM Tris, pH 7.5 electrolyte. (C, D) Continuous current vs. time traces for MspA M2 (C) and sc₈MspA_{dt} M2 (D) in 1M KCl, 2 M GdmCl, 10 mM Tris, pH 7.5 buffer with 75 mV voltage applied, showing the translocation events caused by DNA hairpin poly-dT going through the nanopore. (E) Continuous current vs. time traces for sc₈MspA_{dt} M2 in 1M KCl, 10 mM Tris, pH 7.5 buffer with 140 mV voltage applied. (F, G) Histograms of current change (ΔI) caused by the blockade of individual DNA hairpins(top panel) and DNA hairpin mixture (bottom panel) for MspA M2 (F) and sc₈MspA_{dt} M2 (G) in 1 M KCl, 2 M GdmCl, 10 mM Tris, pH 7.5 electrolyte and 75 mV voltage. 0.3 μM of each DNA hairpin (poly-dA, poly-dC, poly-dG and poly-dT) were added.

FIGS. 5A-C shows plasmids for expression of single-chain mspA genes in E. coli. (A) sc₈mspA m2—single-chain MspA M2 with eight subunits, (B) sc₈mspA pn1—single-chain MspA PN1 with eight subunits, (C) sc₈mspA_{dt} m2-single-chain MspA M2 with eight subunits, an N-terminal histidine tag and a C-terminal TwinStrepII-tag. bla, ampicillin resistance gene; Sm, streptomycin resistance; Ori, origin of replication for E. coli; lacI, lac repressor protein; T7 P, T7 promoter; SD, Shine-Dalgarno sequence; m2-1-m2-8, mspA M2 codon optimized genes; m2-97F-1-m2-97F-8, codon-optimized genes encoding MspA M2 with P97F mutation; His-tag—histidine tag, StrepII−TwinStrepII tag, TEV—TEV cleavage site. Note, that linkers connecting adjacent mspA M2 genes are not shown on the maps. Cloning of pML3216 and pML3222: The parent vectors pML3215 and pML3221 carry sc₈mspA m2, and sc₈mspA pn1 genes, respectively, in the pUC57 background. The genes were synthesized by GenScript. The plasmids were digested with EcoRI and HindIII to obtain the single-chain mspA genes, which were then cloned into an appropriately digested pET-21(a)+vector to produce plasmids pML3216 and pML3222, which were used for protein production in E. coli. To obtain pML4170 the first m2-1 gene encoding MspA M2 with an N-terminal histidine-tag and the last m2-8 gene encoding MspA M2 with a C-terminal TwinStrepII tag were ordered from GenScript. These genes were subsequently cloned into pML3216. Then, the gene encoding all eight MspA subunits with N- and C-terminal tags was excised and cloned into pCDFDuet-1 backbone to give pML4170.

FIGS. 6A-B show cloning of single-chain mspA genes with non-octameric stoichiometry. (A) Plasmid designations are given in gray boxes. The primers used for PCR amplification are presented in light boxes. Oligonucleotide sequences are shown in Table 4. Parent vectors are shown in boxes along with the restriction sites that were used for digestion. Green boxes indicate restriction enzymes used for the digestion of a backbone plasmid or an insert fragment for subsequent ligation. Plasmids pML3227, pML3228, pML3229, pML3230 carry hepta-, hexa-, penta-, tri-meric constructs in pUC57 background, respectively, shown in B. Plasmids pML3231, pML3232, pML3233, pML3234 were obtained by excising single-chain genes with EcoRI and HindIII and inserting the genes into pET-21(a)+background for protein production. (B) single-chain mspA genetic constructs: i) heptameric single-chain—sc₇mspA pn1; ii) hexameric single-chain—sc₆mspA pn1; iii) pentameric single-chain—sc₅mspA pn1; iv) trimeric single-chain—sc₃mspA pn1. Numbered arrows represent translationally fused mspA pn1 genes. Blue boxes show linker regions connecting adjacent mspA genes. Restriction sites at the beginning and end of each gene are shown below and above the plane of scheme, respectively. The scheme is not to scale. Each construct has TGA stop-codon in front of HindIII restriction site.

FIG. 7 shows production of single-chain MspA variants with different subunit stoichiometries in E. coli. Approximately 100 ng of protein in whole-cell lysates of E. coli Omp8 was loaded onto 10% polyacrylamide gel followed by Coomassie stain. − and +signs represent absence or presence of 1.5 mM IPTG in the culture medium, respectively. Lanes: M—molecular weight marker with sizes in kDa shown on the left; sc₇MspA—expression of sc₇MspA PN1 with 7 covalently linked subunits (pML3231); sc₆MspA—expression of sc₆MspA PN1 with 6 covalently linked subunits (pML3232); sc₅MspA—expression of sc₅MspA PN1 with 5 covalently linked subunits (pML3233); sc₃MspA—expression of sc₃MspA PN1 with 3 covalently linked subunits (pML3234).

FIGS. 8A-E show histograms of channel conductances of octameric and single-chain MspA pores with eight subunits. Recordings were performed in 1 M KCl, 10 mM HEPES, pH 7.4 electrolyte at −10 mV applied potential. Diameter of the diphytanoyl phosphatidylcholine (DPhPC) bilayer was approximately 1 mm. All protein samples were added to the both sides of the cuvette (A) wtMspA—octameric MspA wild-type protein from M. smegmatis—94 insertions from 5 membranes are plotted; (B) MspA M1—octameric MspA M1 protein from M. smegmatis—105 insertions from 3 membranes; (C) MspA M2-octameric MspA M2 protein from M. smegmatis—186 insertions from 9 membranes; (D) sc₈MspA M2—single-chain MspA M2 with eight covalently linked subunits, 175 insertions from 9 membranes; (E) sc₈MspA_{dt} M2—in diphytanoyl phosphatidylcholine (DPhPC) bilayer, 432 insertions from 8 membranes; (F) double-tagged single-chain MspA M2 with eight covalently linked subunits in bi-block polymer (PBD-PEO) bilayer, a total of 207 insertions from 28 different membranes plotted.

FIGS. 9A-F show histograms of channel conductances of single-chain MspA pores with different subunit stoichiometries. Recordings were performed in 1 M KCl, 10 mM HEPES, pH 7.4 electrolyte at −10 mV applied potential. Diameter of the diphytanoyl phosphatidylcholine (DPhPC) bilayer was approximately 1 mm. All protein samples were added to the both sides of the cuvette. (A) MspA PN1—octameric MspA PN1 (D90N/D91N/D93N/P97F/D118R/D134R/E139K) protein from M. smegmatis—270 insertions from 3 membranes are plotted; (B) sc₃MspA PN1—95 insertion events from 4 membranes were recorded; (C) sc₅MsA PN1—221 insertions from 7 membranes; (D) sC₆MspA PN1—146 insertion events from 6 different membranes; (E) sc₇MspA PN1—62 insertions from 5 membranes; (F) sc₈MspA PN1—244 insertions from 8 membranes.

FIG. 10 shows contamination and degradation of single-chain MspA proteins purified by gel excision protocol. Polyacrylamide gel with sc₈MspA PN1 after gel excision and elution (GE) and refolding (RE). Lower molecular bands marked with asterisks are present. These bands are contaminants and/or degradation products resulting from linker proteolysis of single-chain MspA protein.

FIGS. 11A-D show sc₈MspA_{dt} M2 purification. (A) Workflow steps. Two step sequential affinity purification ensures that only full-length protein is purified. Asterisk denotes purification step in denaturing conditions (8 M urea). (B) Polyacrylamide gel showing purification progress from workflow in (A). Lanes: L—molecular weight marker with masses in kDa on the left, IB—inclusion bodies fraction; Ni—Ni-affinity purification on Ni-NTA resin; GF—gel filtration step on Superdex S200 HiLoad 26/60 column; ST—StretII tag-affinity purification on StrepTactin XT resin; RF—sample after refolding by dialysis. (C) Chromatogram of final polishing gel filtration of refolded sample on SuperdesxS 200 Increase 15/30 column. (D) Polyacrylamide gel with fractions from c. Lanes: L—molecular weight marker with masses in kDa on the left, 18-31—fractions from gel filtration shown in c.

FIGS. 12A-B show characterization of sc₈MspA_{dt} M2 in single channel experiments. (A) Current vs. time traces for sc₈MspA_{dt} M2. Gating with different current levels always happens when negative voltage is applied. For correction, the current value was chosen as shown by the red dashed line for the I-V curves. (B) Current vs. Voltage curves for uncorrected and corrected baseline levels. All data were obtained in 1M KCl, 2 M GdmCl, 10 mM Tris, pH 7.5 buffer.

FIGS. 13A-H show current traces of MspA M2 and sc₈MspA_{dt} M2 with different hairpin constructs. Current traces for a single channel insertion of MspA M2 or sc₈MspA_{dt} with 0.4 μM of poly-dA (A, B), poly-dC (C, D), poly-dG3dA (E, F), and mixture of 0.3 μM poly-dA, 0.3 μM poly-dC, 0.3 μM poly-dG3dA, and 0.3 μM poly-dT (G, H) hairpins. All experiments were performed in 1 M KCl, 2 M GdmCl, 10 mM Tris, pH 7.5 buffer with 75 mV bias applied to the cis chamber.

FIGS. 14A-D show electron microscopy of single-chain MspA (scMspA) constructs. A. A representative negatively stained electron micrograph of sc8MspA M2 from pML4170 (stain: 1% uranyl formate). Spherical protein aggregates are visible. B. A representative negatively stained (1% uranyl formate) electron micrograph of sc8MspA M2 from pML4173. Note the absence of spherical protein aggregates. Images similar to B were used to make 2D class averages of shown in C and D. C, D. Electron microscopy class average from 2,895 sc8MspA M2 particles produced from pML4173, top (C) and cross-section (D) views (stain: 1% uranyl formate, scale bars 10 nm).

FIGS. 15A-E show Characterization of single-chain MspA with acidic linkers. A. Model of the “octamer-like” sc₈MspA M2. N-terminus has His8 tag and C-terminus has Twin-Strep II tag for purification. TEV protease recognition sites are shown. All eight msp genes are connected by linkers to form scMspA (below pML4173). Scheme is not to scale. B. Plasmid for expression of single-chain MspA with acidic linkers. m2-1-m2-8: mspA M2 codon optimized genes with mutations D90N/D91N/D93N//D118R/D134R/E139K in all eight genes; Sm: streptomycin resistance; Ori: origin of replication for E. coli; lac: lac repressor gene; T7 P: T7 promoter; SD: Shine-Dalgarno sequence;. His-tag: histidine tag, StrepII: TwinStrepII tag, TEV: TEV cleavage site. Note that linkers connecting adjacent mspA M2 genes are not shown in the maps. C. Purification of scMspA. Lanes: IB—inclusion bodies purified from E. coli; Ni—scMspA after Ni-affinity purification; G1—scMspA sample after gel filtration; Str—scMspA sample after StrepII tag-affinity purification; Ref—scMspA after refolding by dialysis; G2—sample after final gel filtration purification, fraction shown here was used for lipid bilayer experiments in D and E. Numbers represent molecular weights of the marker in kDa. D. Current traces of sc8MspA M2 in a DPhPC membrane with an aperture of 1 mm in the Montal-Mueller system recorded at a bandwidth of 33 Hz, showing insertion activity at 16 ng/ml. Recordings were done at −10 mV in 1 M KCl, 10 mM HEPES, pH 7.4 buffer. E. Distribution histogram of 181 opening events recorded from 3 different membranes. Broad distribution shown with predominant peak at 1.6 nS. Concentration of protein was 16 ng/ml in all three membranes.

### DETAILED DESCRIPTION

Provided herein are compositions, for example, nucleic acid constructs encoding a single chain Msp (e.g., MspA) comprising at least two Msp (e.g., MspA) monomers, and purification methods to produce a purified population of single chain Msps (e.g., MspAs). In some instances, one or more of the MspA monomers of the single chain MspA comprise asymmetric mutations to alter the MspA pore properties for specific applications. As shown in the Examples herein, single-chain MspA trimers, pentamers, hexamers, heptamers and octomers were constructed to provide pores with different channel diameters by controlling their subunit stoichiometry. All single-chain MspA proteins formed functional channels in lipid bilayer experiments. Importantly, full-length single-chain MspA discriminated all four nucleotides in a manner identical to MspA produced from monomers.

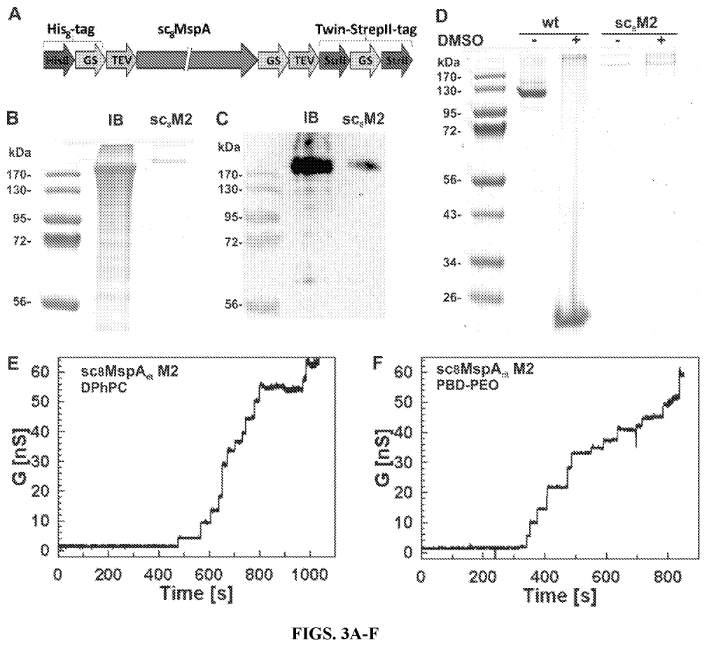

Provided herein is a method for preparing a purified population of single chain Msps, (for example, MspAs). The method comprises (a) expressing in E. coli a polypeptide comprising a single chain MspA, wherein the polypeptide comprises, in the following order, (i) a first affinity tag, wherein the affinity tag is a polyhistidine tag; (ii) a first amino acid linker; (iii) a single chain MspA, wherein the single chain MspA comprises at least a first MspA monomer sequence and a second MspA monomer sequence; wherein the first and second monomer sequence are linked by a second amino acid linker; (iv) a third amino acid linker; and (v) a second affinity tag; (b) recovering inclusion bodies that express the single chain MspAs from the E. coli under denaturing conditions; (c) using Ni-affinity chromatography to obtain one or more fractions comprising single chain MspAs from the inclusion bodies under denaturing conditions; (d) optionally separating the single chain MspAs in the one or more fractions using size exclusion chromatography to obtain a desired fraction comprising MspAs; (e) purifying the MspAs from the one or more fractions of step (c) or the desired fraction of step (d) using second affinity tag purification under denaturing conditions; (f) refolding the purified MspAs of step (e) in a refolding buffer, wherein the refolding buffer comprises about 50 mM to about 200 mM L-Arginine, about 800 mM to about 1000 mM urea, and 0.5% to about 1.0% OPOE, wherein the buffer has a pH of about 7.0 to about 8.5; and (g) concentrating the refolded MspAs using size exclusion chromatography to obtain a purified population of single chain MspAs. FIG. 3A provides an exemplary construct (i.e., a polypeptide comprising a single chain MspA, wherein the polypeptide comprises, in the following order, (i) a first affinity tag, wherein the affinity tag is a polyhistidine tag; (ii) a first amino acid linker; (iii) a single chain MspA, wherein the single chain MspA comprises at least a first MspA monomer sequence and a second MspA monomer sequence; wherein the first and second monomer sequence are linked by a second amino acid linker; (iv) a third amino acid linker; and (v) a second affinity tag) for use in the the purification methods described herein.

The single-chain Msps described herein, e.g., MspAs, are expressed in cells, such as bacterial cells, and then purified from inclusion bodies. The single-chain Msps purified from inclusion bodies are then refolded using the steps as described herein.

The term expression or expressing refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity, e.g. porin activity. Also, the process of expression may involve further processing steps to the RNA product of transcription, such as splicing to remove introns, and/or post-translational processing of a polypeptide product.

In the methods described herein, a vector comprising a nucleic acid encoding a polypeptide described herein is transfected into a host cell, e.g., E. coli. The vector can further comprise a promoter sequence, for example, a constitutive promoter or an inducible promoter. Examples of constitutive promoters include, but are not limited to, the p_{smyc} promoter and Phsp60. Examples of inducible promoters include, but are not limited to, an acetamide-inducible promoter and a tetracycline inducible promoter. In some methods, the promoter is a T7 promoter.

Any of the single-chain Msps disclosed herein can be produced by transforming a mutant bacterial strain comprising a deletion of a wild-type MspA, a wild-type MspB, a wild-type MspC, a wildtype MspD, with a vector comprising an inducible promoter operably linked to a nucleic acid sequence encoding the single-chain Msp porin; and purifying the single-chain Msp porin as described herein (See, for example, U.S. Pat. No. 6,746,594 incorporated herein by reference). Optionally, the mutant bacterial strain comprises a deletion of a recA gene. Optionally, the vector comprises any of the nucleic acids encoding a single-chain Msp described herein. The bacterial strain can further comprise M. smegmatis strain ML16, ML714 or ML712. Optionally, A Mycobacterium smegmatis strain free of endogenous porins is also contemplated for use in the methods provided herein, and can further comprise any vector described herein. By “free” is meant that an endogenous porin cannot be detected in an immunoblot when using an appropriate Msp-specific antiserum, or comprising less than 1% endogenous porins.

Methods for preparing and transforming bacteria, for example in E. coli, with a nucleic acid encoding a polypeptide are known in the art. See, for example, Sambrook et al. Molecular Cloning: A Laboratory Manual, Cold Spring Harbor Laboratory Press (2001). In some methods, the single-chain polypeptide is expressed in E. coli BL21(DE3)Omp8 strain which lacks 3 major porins (See Prilipov et al. FEMS Microbiol. Lett 163: 65-72 (1998). Methods for preparing and extracting insoluble (i.e., inclusion-body) proteins from E. coli, are known in the art. See, for example, Palmer and Wingfield “Preparation and Extraction of Insoluble (Inclusion-body) Proteins from E. coli,” Curr. Protoc. Protein Sci. Chapter: Unit-6.3 (2004)). Upon expression in bacteria, the single-chain Msps, (e.g., MspAs) accumulate in inclusion bodies. As used herein, inclusion bodies are typically dense, spherical, aggregated proteins, that are mostly formed in the cytoplasm of prokaryotes due to overexpression of heterologous proteins. [symbol] See also, the Examples below, for methods of recovering inclusion bodies and purifying single-chain Msps from inclusion bodies. In the methods provided herein, inclusion bodies are recovered under denaturing conditions. Typically, denaturation involves the breaking of weak linkages, or bonds (e.g., hydrogen bonds), within a protein molecule that are responsible for the highly ordered structure of the protein in its natural (native) state. Denatured proteins generally have a looser, more random structure, and in some cases, are insoluble. As used herein, denaturing conditions can comprise one or more of heat, mechanical agitation, pH changes to disrupt salt bridges, ureal/chaotropic agents, nonpolar solvents, detergents or heavy metals.

In the methods described herein, after recovery of the inclusion bodies, nickel affinity chromatography is used to obtain one or more fractions (e.g., one, two, three, four, five, six or more fractions) comprising single-chain MspAs from the inclusion bodies. In nickel affinity chromatography, nickel columns are used for immobilized metal affinity chromatography (IMAC) for the purification of recombinant proteins with a polyhistidine tag (e.g., the first affinity tag) on either terminus of a polypeptide.

Following Ni-affinity purification, the methods provided herein optionally comprise a step of separating the single chain MspAs in the one or more fractions (e.g., one, two, three, four, five, six or more fractions obtained using Ni-affinity purification), using one or more of precipitation, centrifugation, depth filtration, affinity chromatography, size exclusion chromatography, ion exchange chromatography, mixed mode anion exchange chromatography, or hydrophobic interaction chromatography, to obtain one or more desired fractions (e.g., one, two, three, four, five, six or more desired fractions) comprising MspAs.

In some methods, size exclusion chromatography is optionally used to obtain one or more desired fractions after Ni-affinity chromatography. In some methods, one or more size-exclusion chromatagraphy steps can be performed on any one or more fractions obtained using Ni-affinity chromatography. Some methods further comprise taking samples during the purification process, evaluating the samples to quantitatively and/or qualitatively monitor characteristics of the recombinant MspAs and the purification process. In some methods, the samples are quantitatively and/or qualitatively monitored using process analytical techniques.

In any of the methods described herein, the single-chain Msps can be purified from (i) the one or more fractions obtained using Ni-affinity chromatography or (ii) one or more desired fractions obtained after size exclusion chromatography, ion exchange chromatography, mixed mode anion exchange chromatography, hydrophobic interaction chromatography or hydroxyapatite chromatography, using second affinity tag purification under denaturing conditions.

As used herein, the term purified or purify refers to separating a substance, e.g., single-chain Msp(s) from at least some of the components (e.g., impurities or contaminants) with which it was associated when initially produced. For example, single-chain Msp(s) are purified by removal of cellular components, contaminating proteins and nucleic acid species, to name a few. Purified substances can be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated.

As used herein, the term refolding refers to the process under which a protein, for example a protein isolated from inclusion bodies, is folded into its characteristic and functional three-dimensional structure from a prior random orientation, for example, its orientation after recovery from inclusion bodies, for example, under denaturing conditions.

In some methods, the refolding buffer comprises between about 50 to about 200 mM L-Arginine, about 800 to about 1000 mM urea, and about 0.5% to about 1% OPOE, wherein the buffer has a pH of about 7.5 to about 8.0. In some methods, the refolding buffer comprises between about 50 to about 200 mM L-Arginine, about 800 to about 1000 mM urea, about 0.5% to about 1% OPOE, and about 150 mM to about 500 mM NaCl, wherein the buffer has a pH of about 7.5 to about 8.0. In some methods, the refolding buffer comprises between about 50 to about 200 mM L-Arginine, about 800 to about 1000 mM urea, about 0.5% to about 1% OPOE, about 150 mM to about 500 mM NaCl, and about 25 mM to about 50 mM inorganic phosphates (e.g., a sodium inorganic phosphate (NaPi)), wherein the buffer has a pH of about 7.5 to about 8.0 In some methods, the refolding buffer comprises about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM L-arginine. In some methods, the refolding buffer comprises between about 50 mM to about 210 mM, about 60 mM to about 210 mM, about 70 mM to about 210 mM, about 80 mM to about 210 mM, about 90 mM to about 210 mM, about 100 mM to about 210 mM, about 105 mM to about 210 mM, about 110 mM to about 210 mM, about 115 mM to about 210 mM, about 120 mM to about 210 mM, about 130 mM to about 210 mM, about 140 mM to about 210 mM, about 150 mM to about 210 mM, about 160 mM to about 210 mM, about 170 mM to about 210 mM, about 180 mM to about 210 mM, about 185 mM to aboute 210 mM, about 190 mM to about 210 mM, about 195 mM to about 210 mM, about 200 mM to about 210 mM, 50 mM to about 200 mM, about 60 mM to about 200 mM, about 70 mM to about 200 mM, about 80 mM to about 200 mM, about 90 mM to about 200 mM, about 100 mM to about 200 mM, about 105 mM to about 200 mM, about 110 mM to about 200 mM, about 115 mM to about 200 mM, about 120 mM to about 200 mM, about 130 mM to about 200 mM, about 140 mM to about 200 mM, about 150 mM to about 200 mM, about 160 mM to about 200 mM, about 170 mM to about 200 mM, about 180 mM to about 200 mM, about 185 mM to aboute 200 mM, about 190 mM to about 200 mM, or about 195 mM to about 200 mM L-arginine.

In some methods, the refolding buffer comprises about 750 mM, 760 mM, 770 mM, 780 mM, 790 mM, 800 mM, 810 mM, 820 mM, 830 mM, 840 mM, 850 mM, 860 mM, 870 mM, 880 mM, 890 mM, 900 mM, 910 mM, 920 mM, 930 mM, 940 mM, 950 mM, 960 mM, 970 mM, 980 mM, 990 mM, or about 1000 mM urea. In some embodiments, the refolding buffer comprises about 750 mM to about 850 mM, about, 760 mM to about 850 mM, about 770 mM to about 850 mM, about 780 mM to about 850 mM, about 790 mM to about 850 mM, about 800 mM to about 850 mM, about 810 mM to about 850 mM, about 820 mM to about 850 mM, about 830 mM to about 850 mM, about 840 mM to about 850 mM, 750 mM to about 800 mM, about, 760 mM to about 800 mM, about 770 mM to about 800 mM, about 780 mM to about 800 mM, about 790 mM to about 800 mM, about 800 mM to about 900 mM, about 810 mM to about 900 mM, about 820 mM to about 900 mM, about 830 mM to about 900 mM, about 840 mM to about 900 mM, about 850 mM to about 900 mM, about 860 mM to about 900 mM, about 870 to about 900 mM, about 880 to about 900, about 890 mM to about 900 mM, about 800 mM to about 1000 mM, about 810 mM to about 1000 mM, about 820 mM to about 1000 mM, about 830 mM to about 1000 mM, about 840 mM to about 1000 mM, about 850 mM to about 1000 mM, about 860 mM to about 1000 mM, about 870 to about 1000 mM, about 880 to about 1000, about 890 mM to about 1000 mM, about 900 mm to about 1000 mM, about 910 mM, to about 1000 mM, about 920 mM to about 1000 mM, about 930 mM to about 1000 mM, about 940 mM to about 1000 mM, about 950 mM to about 1000 mM, about 960 mM to about 1000 mM, about 970 to about 1000 mM, about 980 to about 1000, or about 990 mM to about 1000 mM urea.

In some embodiments, the refolding buffer comprises about 0.2% to about 1%, about 0.25% to about 1%, about 0.30% to about 1%, about 0.35% to about 1%, about 0.4% to about 1%, about 0.45% to about 1%, about 0.50% to about 1%, about 0.55% to about 1%, about 0.60% to about 1%, about 0.65% to about 1%, about 0.7% to about 1%, about 0.75% to about 1%, about 0.8% to about 1%, about 0.85% to about 1%, about 0.9% to about 1%, or about 0.95% to about 1% octyl polyoxyethylene (OPOE).

In some methods, the refolding buffer comprises about 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, or 500 mM of a sodium salt (e.g., NaCl). In some methods, the refolding buffer comprises about 150 mM to about 500 mM of a sodium salt, (e.g., NaCl), about 160 mM to about 500 mM, about 170 mM to about 500 mM, about 180 mM to about 500 mM, about 190 mM to about 500 mM, about 200 mM to about 500 mM, about 210 mM to about 500 mM, about 220 mM to about 500 mM, about 230 mM to about 500 mM, about 240 mM to about 500 mM, about 250 mM to about 500 mM, about 260 mM to about 500 mM, about 270 mM to about 500 mM, about 280 mM to about 500 mM, about 290 mM to about 500 mM, about 300 mM to about 500 mM, about 310 mM to about 500 mM, about 320 mM to about 500 mM, about 330 mM to about 500 mM, about 340 mM to about 500 mM, about 350 mM to about 500 mM, about 360 mM to about 500 mM, about 370 mM to about 500 mM, about 380 mM to about 500 mM, about 390 mM to about 500 mM, about 400 mM to about 500 mM, about 410 mM to about 500 mM, about 420 mM to about 500 mM, about 430 mM to about 500 mM, about 440 mM to about 500 mM, about 450 mM to about 500 mM, about 460 mM to about 500 mM, about 470 mM to about 500 mM, about 480 mM to about 500 mM, or about 490 mM to about 500 mM of a sodium salt (e.g., NaCl).

In some methods, the refolding buffer comprises about 150 mM to about 300 mM of a sodium salt, (e.g., NaCl), about 160 mM to about 300 mM, about 170 mM to about 300 mM, about 180 mM to about 300 mM, about 190 mM to about 300 mM, about 200 mM to about 300 mM, about 210 mM to about 300 mM, about 220 mM to about 300 mM, about 230 mM to about 300 mM, about 240 mM to about 300 mM, about 250 mM to about 300 mM, about 260 mM to about 300 mM, about 270 mM to about 300 mM, about 280 mM to about 300 mM, or about 290 mM to about 300 mM NaCl.

In some methods, the refolding buffer comprises about 150 mM to about 350 mM, about 160 mM, to about 350 mM, about 170 mM to about 350 mM, about 180 mM to about 350 mM, about 190 mM to about 350 mM, about 200 mM to about 350 mM, about 210 mM, to about 350 mM, about 220 mM to about 250 mM, about 230 mM to about 350 mM, about 240 mM to about 350 mM, about 250 mM to about 350 mM, about 260 mM, to about 350 mM, about 270 mM to about 350 mM, about 280 mM to about 350 mM, about 290 mM to about 350 mM, or about 300 mM to about 350 mM NaCl.

In some methods, the refolding buffer comprises 25 mM to about 50 mM inorganic phosphates (e.g., NaPi), about 30 mM to about 50 mM, about 35 mM to about 50 mM, about 40 mM to about 50 mM, about 45 mM to about 50 mM, about 25 mM to about 45 mM, about 25 mM to about 40 mM, about 25 mM to about 35 mM, or about 25 mM to about 30 mM inorganic phosphates.

In some embodiments, the pH of the refolding buffer is about 7.0 to about 8.5, about 7.1 to about 8.5, 7.2 to about 8.5, 7.3 to about 8.5, 7.4 to about 8.5, 7.5 to about 8.5, 7.6 to about 8.5, 7.7 to about 8.5, about 7.8 to about 8.5, about 7.9 to about 8.5, about 8.0 to about 8.5, about 8.1 to about 8.5, about 8.2 to about 8.5, about 8.3 to about 8.5, or about 8.4 to about 8.5. In some embodiments, the pH of the refolding buffer is about 7.0, 7.1, 7.2, 7.3, 7.4, 7.5, 7.6, 7.7, 7.8, 7.9, 8.0, 8.1, 8.2, 8.3, 8.4, or 8.5.

In some embodiments, the refolding buffer comprises about 150 mM to about 500 mM NaCl, about 25 to about 50 mM NaPi, about 50 mM to about 200 mM L-Arginine, about 800 mM to about 1000 mM urea, about 0.5% to about 1.0% octyl polyoxyethylene (OPOE), about 0.5 mM to about 1 mM phenylmethylsulfonyl fluoride (PMSF), one or more protease inhibitors (for example, one or more aminopeptidase inhibitors, metalloprotease inhibitors, serine protease inhibitors, cystein protease inhibitors or aspartic acid protease inhibitors), and about 0.02% sodium azide, wherein the buffer has a pH of about 7.0 to about 8.5. In some embodiments the pH of the refolding buffer is about 8.0.

Optionally, in any of the methods provided herein, the MsPs, e.g., MspAs, comprise (a) a first MspA monomer sequence; (b) a second MspA monomer sequence; and (b) a third, fourth, fifth, sixth, seventh, and eighth MspA monomer sequence or any subset thereof, wherein the first, second, third, fourth, fifth, sixth, seventh and eighth MspA monomer sequence or any subset thereof are arranged consecutively and wherein the second amino acid linker is positioned between any two Msp monomer sequences. In some methods, the second amino acid linker is positioned between every two Msp monomer sequences. In some methods, the polypeptide comprises a first second, third, fourth, fifth, sixth, seventh, eighth MspA monomer sequence, wherein the second amino acid linker is positioned between the first and second Msp monomer, second and third Msp monomer, third and fourth Msp monomer, fourth and fifth Msp monomer, fifth and sixth Msp monomer, sixth and seventh Msp monomer, and seventh and eighth Msp monomer. It is understood that the second amino acid linker positioned between any two monomers in the MspA can be the same or different from another second amino acid linker sequence in the single-chain MspA. For example, a second amino acid linker positioned between the first and second monomer in the MspA can be the same or different as a second amino acid linker positioned between the second and third monomer, the same or different from a second amino acid linker positioned between the third and fourth monomer of the MspA, etc.

In some embodiments, one or more second amino acid linkers that separate two or more monomers in the MspA are amino acid linkers having a net charge, at pH 7.0, of about 0.1 to about −5.0. In some embodiments, one or more second amino acid linkers that separate two or more monomers in the MspA are acidic amino acid linkers that have a negative charge at pH 7.0, for example, an acidic amino acid linker having a net charge, at pH 7.0, of about −2.0 to about −5.0. For example, the linker can have a net charge of about −2.1 to about −5.0, about −2.2 to about −5.0, about −2.3 to about −5.0, about −2.4 to about about −5.0, about −2.5 to about −5.0, about −2.6 to about −5.0, about −2.7 to about −5.0, about −2.8 to about −5.0, about −2.9 to about −5.0, about −3.0 to about −5.0, about −3.1 to about −5.0, about −3.2 to about −5.0, about −3.3 to about −5.0, about −3.4 to about −5.0, about −3.5 to about −5.0, about −3.6 to about −5.0, about −3.7 to about −5.0, about −3.8 to about −5.0, about −3.9 to about −5.0, or about −4.0 to about −5.0. For example, the linker can have a net charge of about −1, −1.1, −1.2, −1.3, −1.4, −1.5, −1.6, −1.7, −1.8, −1.9, −2, −2.1, −2.2, −2.3, −2.4, −2.5, −2.6, −2.7, −2.8, −2.9, −3.−3.1, −3.2, −3.3, −3.4, −3.5, −3.6, −3.7, −3.8, −3.9, −4.0, −4.1, −4.2, −4.3, −4.4, −4.5, −4.6, −4.7, −4.8, −4.9, or −5.0. Exemplary acidic amino acid linkers that can be used, include but are not limited to those set forth in Table 6 and Table 7. In some examples, the single-chain MspA contains one or more acidic amino acid linkers comprising SEQ ID NO: 53, SEQ ID NO: 54. SEQ ID NO: 55, SEQ ID NO: 56, SEQ ID NO: 57, SEQ ID NO: 58, or SEQ ID NO: 59. In some examples, the single-chain MspA contains one or more amino acid linkers comprising SEQ ID NO: 23, SEQ ID NO: 25, SEQ ID NO: 27, SEQ ID NO: 29, SEQ ID NO: 31, SEQ ID NO: 33, or SEQ ID NO: 35. In some examples, the single-chain MspA contains one or more acidic amino acid linkers comprising SEQ ID NO: 60, SEQ ID NO: 61. SEQ ID NO: 62, SEQ ID NO: 63, SEQ ID NO: 64, SEQ ID NO: 65, or SEQ ID NO: 66. Amino acid linkers having at least 95% sequence identity with any one of SEQ ID NOs: 23, 25, 27, 29, 31, 33, 35, 53, 54, 55, 56, 57, 58, 59, 60, 61, 62, 63, 64, 65, or 66 can also be used in any of the constructs or methods described herein.

In some embodiments, SEQ ID NO: 53 separates the first and second monomer of the MspA, SEQ ID NO: 54 separates the second and third monomer of the MspA, SEQ ID NO: 55 separates the third and fourth monomer of the MspA, SEQ ID NO: 56 separates the fourth and fifth monomer of the MspA, SEQ ID NO: 57 separates the fifth and sixth monomer of the MspA, SEQ ID NO: 58 separates the sixth and seventh monomer of the MspA, and SEQ ID NO: 59 separates the seventh and eighth monomer of the MspA.

In some embodiments, SEQ ID NO: 60 separates the first and second monomer of the MspA, SEQ ID NO: 61 separates the second and third monomer of the MspA, SEQ ID NO: 62 separates the third and fourth monomer of the MspA, SEQ ID NO: 63 separates the fourth and fifth monomer of the MspA, SEQ ID NO: 64 separates the fifth and sixth monomer of the MspA, SEQ ID NO: 65 separates the sixth and seventh monomer of the MspA, and SEQ ID NO: 66 separates the seventh and eighth monomer of the MspA.

In some embodiments, SEQ ID NO: 23 separates the first and second monomer of the MspA, SEQ ID NO: 25 separates the second and third monomer of the MspA, SEQ ID NO:27 separates the third and fourth monomer of the MspA, SEQ ID NO: 29 separates the fourth and fifth monomer of the MspA, SEQ ID NO: 31 separates the fifth and sixth monomer of the MspA, SEQ ID NO: 33 separates the sixth and seventh monomer of the MspA, and SEQ ID NO: 35 separates the seventh and eighth monomer of the MspA.

As described in the Examples, the number of Msp monomers in the single-chain Msp can be varied, to modulate the diameter and/or the conductance of the Msps. In some cases, the Msps produced by any of the methods provided herein have a conductance of about 0.5 nanosiemens (nS) to about 6 nS. For Example, the Msps can have a conductance of about 0.5 nS to about 6 nS, about 0.6 to about 6 nS, about 0.7 nS to about 6 nS, about 0.8 nS to about 6 nS, about 0.9 nS to about 6 nS, about 1.0 nS to about 6.0 nS, about 1.5 nS to about 6 nS, about 2.0 nS to about 6 nS, about 2.5 nS to about 6 nS, about 3.0 nS to about 6 nS, about 3.5 nS to about 6.0 nS, about 4.0 nS to about 6.0 nS, about 4.5 nS to about 6.0 nS, about 5.0 nS to about 6.0 nS, or about 5.5 to about 6.0 nS, about 0.5 nS to about 5 nS, about 0.6 to about 5 nS, about 0.7 nS to about 5 nS, about 0.8 nS to about 5 nS, about 0.9 nS to about 5 nS, about 1.0 nS to about 5 nS, about 1.5 nS to about 5 nS, about 2.0 nS to about 5 nS, about 2.5 nS to about 5 nS, about 3.0 nS to about 5 nS, about 3.5 nS to about 5 nS, about 4.0 nS to about 5 nS, about 4.5 nS to about 5 nS, 0.5 nS to about 4 nS, about 0.6 to about 4 nS, about 0.7 nS to about 4 nS, about 0.8 nS to about 4 nS, about 0.9 nS to about 4 nS, about 1.0 nS to about 4 nS, about 1.5 nS to about 4 nS, about 2.0 nS to about 4 nS, about 2.5 nS to about 4 nS, about 3.0 nS to about 4 nS, or about 3.5 nS to about 4 nS, 0.5 nS to about 3 nS, about 0.6 to about 3 nS, about 0.7 nS to about 3 nS, about 0.8 nS to about 3 nS, about 0.9 nS to about 3 nS, about 1.0 nS to about 3 nS, about 1.5 nS to about 3 nS, about 2.0 nS to about 3 nS, or about 2.5 nS to about 3 nS, about 0.5 nS to about 2.0 nS, about 1.0 nS to about 2.0 nS, or about 1.5 nS to about 2.0 nS.

In addition to MspA, the methods provided herein can be used to purify other Msp polypeptides, for example, one or more Msp monomers encoded by a gene in Mycobacterium smegmatis. Mycobacterium smegmatis has four identified Msp genes, denoted MspA, MspB, MspC, and MspD. The amino acid sequences for a MspA, MspB, MspC and a MspD monomer without a signal sequence, i.e., the mature portion of the sequence, are provided as SEQ ID NO: 1, SEQ ID NO: 2, SEQ ID NO: 3 and SEQ ID NO: 4, respectively. The amino acid sequences for a MspA, MspB, MspC and a MspD monomer with a signal/leader sequence are provided as SEQ ID NO: 5, SEQ ID NO: 6, SEQ ID NO: 7 and SEQ ID NO: 8, respectively.

Any of the polypeptides described herein can comprise one or more Msp monomer sequences comprising an amino acid sequence that has least 50%, 55%, 60%, 65%, 70%, 75%, 80%, 85%, 90%, 95%, 99% identity or any percentage in between to a polypeptide comprising an amino acid sequence selected from the group consisting of SEQ ID NO: 1-SEQ ID NO: 8. It is also understand that sequence having at least 50%, 55%, 60%, 65%, 70%, 75%, 80%, 85%, 90%, 95%, 99% identity or any percentage in between to a sequence selected from the group consisting of SEQ ID NO: 1-SEQ ID NO: 66 can be used in any of the polypeptides or methods described herein. In some methods, the Msp monomer sequence comprises an amino acid sequence that has least 50%, 55%, 60%, 65%, 70%, 75%, 80%, 85%, 90%, 95%, 99% identity or any percentage in between to SEQ ID NO: 1, i.e., a MspA monomer sequence.

Those of skill in the art readily understand how to determine the identity of two polypeptides or nucleic acids. For example, the identity can be calculated after aligning the two sequences so that the identity is at its highest level. Another way of calculating identity can be performed by published algorithms. Optimal alignment of sequences for comparison can be conducted using the algorithm of Smith and Waterman, Adv. Appl. Math. 2: 482 (1981); by the alignment algorithm of Needleman and Wunsch, J. Mol. Biol. 48: 443 (1970); by the search for similarity method of Pearson and Lipman, Proc. Natl. Acad. Sci. U.S.A. 85: 2444 (1988); by computerized implementations of these algorithms (GAP, BESTFIT, FASTA, and TFASTA in the Wisconsin Genetics Software Package, Genetics Computer Group, 575 Science Dr., Madison, WI; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html); or by inspection.

The same types of identity can be obtained for nucleic acids by, for example, the algorithms disclosed in Zuker, Science 244:48-52, 1989; Jaeger et al. Proc. Natl. Acad. Sci. USA 86:7706-7710, 1989; Jaeger et al. Methods Enzymol. 183:281-306, 1989 that are herein incorporated by this reference for at least material related to nucleic acid alignment. It is understood that any of the methods typically can be used and that, in certain instances, the results of these various methods may differ, but the skilled artisan understands if identity is found with at least one of these methods, the sequences would be said to have the stated identity.

For example, as used herein, a sequence recited as having a particular percent identity to another sequence refers to sequences that have the recited identity as calculated by any one or more of the calculation methods described above. For example, a first sequence has 80 percent identity, as defined herein, to a second sequence if the first sequence is calculated to have 80 percent identity to the second sequence using the Zuker calculation method even if the first sequence does not have 80 percent identity to the second sequence as calculated by any of the other calculation methods. As yet another example, a first sequence has 80 percent identity, as defined herein, to a second sequence if the first sequence is calculated to have 80 percent identity to the second sequence using each of calculation methods (although, in practice, the different calculation methods will often result in different calculated identity percentages).

Further, sequences of wild-type Msp monomers that can be modified are disclosed in GenBank, and these sequences and others are herein incorporated by reference in their entireties as are individual subsequences or fragments contained therein. For example, the nucleotide and amino acid sequences of a wild-type MspA monomer can be found at GenBank Accession Nos. AJ001442 and CAB56052, respectively. The nucleotide and amino acid sequences of a wild-type MspB monomer can be found, for example, at GenBank Accession Nos. NC_008596.1 (from nucleotide 600086 to 600730) and YP 884932.1, respectively. The nucleotide and amino acid sequences of a wild-type MspC monomer can be found, for example, at GenBank Accession Nos. AJ299735 and CAC82509, respectively. The nucleotide and amino acid sequences of a wild-type MspD monomer can be found, for example, at GenBank Accession Nos. AJ300774 and CAC83628, respectively.

As used herein a mutant Msp monomer is an Msp monomer that comprises one or more modifications, relative to the wild-type Msp monomer sequence from which the mutant Msp monomer sequence is derived. A mutant Msp monomer can be a full-length monomer or a functional fragment thereof encoded by a MspA, MspB, MspC or MspD-encoding nucleic acid, for example, an mRNA or a genomic sequence encoding MspA, MspB, MspC or MspD, wherein the monomer comprises one or more modifications.

The amino acids in the Msp proteins described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Williams et al., Mol. Cell. Biol. 9:2574 (1989); Evans et al., J. Amer. Chem. Soc. 112:4011-4030 (1990); Pu et al., J. Amer. Chem. Soc. 56:1280-1283 (1991); Williams et al., J. Amer. Chem. Soc. 113:9276-9286 (1991); and all references cited therein. B and γ amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues of any Msp monomer provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

Nonconservative substitutions, for example, substituting a proline with glycine are also contemplated.

In some Msps, a modification is a mutation. As used throughout, a mutation at a specific amino acid is indicated by the single letter code for the amino acid at a position, followed by the number of the amino acid position in an Msp polypeptide sequence (for example, an amino acid position in SEQ ID NO: 1), and the single letter code for the amino acid substitution at this position. Therefore, it is understood that a P97 mutation is a proline to phenylalanine substitution at amino acid 97 of SEQ ID NO: 1. Similarly, a D90N mutation is an aspartic acid to arginine substitution at amino acid 90 of SEQ ID NO: 1, a D91N mutation is an aspartic to arginine substitution at amino acid 91 of SEQ ID NO: 1, etc. It is also understood that amino acids corresponding to positions in SEQ ID NO: 1 are also provided herein. For example, and not to be limiting, one of skill in the art would understand that, the corresponding amino acid for E139 of SEQ ID NO: 1 in MspB (SEQ ID NO:2), MspC (SEQ ID NO: 3) and MspD (SEQ ID NO: 4) is A139, A139 and K138, respectively.

In some methods, the MspA monomer sequence comprises SEQ ID NO: 1, as set forth below. Any of the polypeptides described herein can comprise an MspA monomer sequence comprising an amino acid sequence that has least 50%, 55%, 60%, 65%, 70%, 75%, 80%, 85%, 90%, 95%, 99% identity or any percentage in between to a polypeptide comprising SEQ ID NO: 1.

(SEQ ID NO: 1)
GLDNELSLVDGQDRTLTVQQWDTFLNGVFPLDRNRLTREWFHSGRAKYI
VAGPGADEFEGTLELGYQIGFPWSLGVGINFSYTTPNILIDDGDITAPP
FGLNSVITPNLFPGVSISADLGNGPGIQEVATFSVDVSGAEGGVAVSNA
HGTVTGAAGGVLLRPFARLIASTGDSVTTYGEPWNMN

In some methods, at least one of MspA monomer sequences of the single-chain Msps is a mutant monomer sequence. In some methods, the mutant monomer sequence comprises a D90N, a D91N, and a D93N mutation. Optionally, in the methods provided herein, any mutant Msp monomer sequence described herein can comprise a mutation at amino acid position D118, a mutation at position D134 or a mutation at position E139. Optionally, a mutation at position E139 can be an E to R (arginine) or an E to K (lysine) substitution. Optionally, a mutation at position D118 can be a D to R substitution or a D to K substitution. Optionally, a mutation at position D134 can be a D to R substitution or a D to K substitution. For example, any mutant Msp monomer sequence described herein can comprise one or more mutations selected from the group consisting of: a D118R mutation, a D134R mutation and a E139K mutation. Optionally, any mutant Msp monomer sequence described herein can further comprise at least one of (i) a mutation at position 93 and (ii) a mutation at position D90, position D91 or both positions D90 and D91. Optionally, the amino acid at position 90, 91 or 93 is substituted with arginine, lysine, histidine, glutamine, methionine, threonine, phenylalanine, tyrosine or tryptophan.

In some methods, the mutant monomer sequence further comprises a P97F mutation. In some methods, a mutant Msp monomer sequence comprising a mutation at position 97 can further comprise (i) a mutation at amino acid position D118, D134 and/or E139 (ii) a mutation at position D93, and/or (iii) a mutation at position D90, position D91 or both positions D90 and D91. For example, a mutant MspA monomer sequence can comprise a D90N mutation, a D91N mutation, a D93N mutation, a P97F mutation, a D118R mutation, a D134R mutation and a E139K mutation. The mutant MspA monomer sequence can also comprise a D90N mutation, a D91N mutation, a D93N mutation, a P97F mutation, a D118R mutation, a D134R mutation and a E139K mutation.

In some examples, the first monomer sequence in the single-chain Msps described herein can be, for example, any wildtype or mutant monomer sequence described herein. For example, the mutant monomer sequence can be a mutant MspA sequence. The second monomer can be selected from the group consisting of a wildtype Msp monomer, a second mutant Msp monomer, a wild-type Msp paralog or homolog monomer, and a mutant Msp paralog or homolog monomer. It is understood that the second mutant Msp monomer can be the same or different than the first mutant Msp monomer. These include, but are not limited to, MspA/Msmeg0965, MspB/Msmeg0520, MspC/Msmeg5483, MspD/Msmeg6057, MppA, PorM1, PorM2, PorM1, Mmcs4296, Mmcs4297, Mmcs3857, Mmcs4382, Mmcs4383, Mjls3843, Mjls3857, Mjls3931 Mjls4674, Mjls4675, Mjls4677, Map3123c, Mav3943, Mvan1836, Mvan4117, Mvan4839, Mvan4840, Mvan5016, Mvan5017, Mvan5768, MUL_2391, Mflv1734, Mflv1735, Mflv2295, Mflv1891, MCH4691c, MCH4689c, MCH4690c, MAB1080, MAB1081, MAB2800, RHA1 ro08561, RHA1 ro04074, and RHA1 ro03127. A wild-type MspA paralog or homolog monomer may be a wild-type MspB monomer.

In some methods, the polypeptide comprises one or more first affinity tags, optionally separated by the first amino acid linker. See, for example, FIG. 3A. In some embodiments, the first affinity tag is positioned at the N-terminus of the polypeptide. The first, second, or third amino acid linker in the Msp polypeptides described herein can comprise 2, 3, 4, 5, 6, 7, 8, 9, 10, 11, 12, 13, 14, 15, 16, 17, 18, 19, 20 or more amino acids. Examples of linkers include but are not limited to GSGₙ wherein n is an integer. Other exemplary linker sequences, for example, a first amino acid linker sequence include, but are not limited to GGGSGGGSGGSA (SEQ ID NO: 9) and GGGSAGGSASGGSAGGGSSA (SEQ ID NO: 10).

Optionally, the first affinity tag is a histidine tag. Polypeptide sequences comprising the first affinity tag (for example, a histidine tag (HHHHHHHH (SEQ ID NO: 11) include, but are not limited to MHHHHHHHHENLYFQGEL (SEQ ID NO: 12), MHHHHHHHHGGGSGGGSGGSAENLYFQEL (SEQ ID NO: 13), and MHHHHHHHHGGGSGGGSGGSAENLYFQGGGSAGGSASGGSAGGGSSAGEL (SEQ ID NO: 14).

In some methods, the polypeptide comprises one or more second affinity tags, optionally separated by the third amino acid linker. Exemplary third amino acid linker sequences include but are not limited to GGGSGGSA (SEQ ID NO: 15) and GGSAGGSASG (SEQ ID NO: 16). The second affinity tag is positioned at the C-terminus of the polypeptide. In some methods, the second affinity tag is a streptavidin tag (for example, WSHPQFEK (SEQ ID NO: 17)). In some methods, the Msp polypeptide comprises two second affinity tags, e.g., streptavidin tags, separate by the third amino acid linker, as shown in FIG. 3A. An exemplary polypeptide sequence encoding the second affinity tag is set forth herein as GGGSGGSAENLYFQGWSHPQFEKSGGSAGGSASGWSHPQFEK (SEQ ID NO: 18). In some methods, the second affinity tag(s) is an epitope tag, a glutathione S-transferase (GST) tag or a Myc tag. In some methods, the second affinity tag(s) is a streptavidin tag, an avidin tag, a polyarginine tag comprising about 5 to about 16 arginine residues, a polyaspartic acid tag comprising about 5 to about 15 aspartic acid residues, a C-terminal or C-tag (e.g., glutamic acid-proline-glutamic acid-alanine (EPEA)), or a Bio-tag (AGKAGEGEIPAPLAGTVSKILVKEGDTVKAGQTVLVLEAMKMETEINAPTDGKVEK VLVKERDAVQGGQGLIKIG) (SEQ ID NO: 21). In some methods, the E. coli is E. coli BL21(DE3) omp8.

In some methods, the polypeptide further comprises a protease cleavage site (for example, ENLYFQ (SEQ ID NO: 20) positioned between the first amino acid linker and the single-chain MspA and/or a protease cleavage site positioned between the third amino acid linker and the second affinity tag. See, FIG. 3A where TEV cleavage sites are shown.

### Polypeptides

Populations or pluralities of Msps (e.g., two or more Msps) produced by any of the methods provided herein are also provided. Any of the Msps produced by the methods provided herein can be inserted in a lipid bilayer for use in, for example, any of the analyte detection methods described herein. Lipid bilayers comprising any of the single-chain Msps produced by the methods described herein are also provided.

Also provided is a polypeptide comprising a single chain MspA, wherein the polypeptide comprises, in the following order, (i) a first affinity tag, wherein the affinity tag is a polyhistidine tag; (ii) a first amino acid linker; (iii) a single chain MspA, wherein the single chain MspA comprises at least a first MspA monomer sequence and a second MspA monomer sequence; wherein the first and second monomer sequence are linked by a second amino acid linker; (iv) a third amino acid linker; and (v) a second affinity tag.

In some polypeptides, the second amino acid linker is positioned between every two Msp monomer sequences. In some polypeptides, the second amino acid linker is an acidic amino acid linker having a net charge of about −2.0 to about −5.0, at pH 7.0. In some polypeptides, the second amino acid linker is selected from the group consisting of SEQ ID NO: 53-SEQ ID NO: 59. In some polypeptides, each MspA monomer sequence has at least 95% identity to SEQ ID NO: 1. In some polypeptides, at least one of MspA monomer sequences is a mutant monomer sequence. In some polypeptides, the mutant monomer sequence comprises a D90N, a D91N, and a D93N mutation. In some polypeptides, the mutant monomer sequence further comprises a D118 mutation, a D134 mutation, and a E139 mutation. In some polypeptides, the mutant monomer sequence further comprises a P97F mutation. In some polypeptides, the mutant monomer sequence comprises a D90N, a D91N, a D93N mutation, a P97F mutation, a D118R mutation, a D134R mutation, and a E139K mutation. In some polypeptides, the single chain MspA comprises at least three MspA monomers. In some polypeptides, the single chain MspA comprises at least five MspA monomers. In some polypeptides, the single chain MspA comprises at least seven MspA monomers. In some polypeptides, the single chain MspA comprises eight MspA monomers.

In some polypeptide, SEQ ID NO: 53 separates the first and second monomer of the MspA, SEQ ID NO: 54 separates the second and third monomer of the MspA, SEQ ID NO: 55 separates the third and fourth monomer of the MspA, SEQ ID NO: 56 separates the fourth and fifth monomer of the MspA, SEQ ID NO: 57 separates the fifth and sixth monomer of the MspA, SEQ ID NO: 58 separates the sixth and seventh monomer of the MspA, and SEQ ID NO: 59 separates the seventh and eighth monomer of the MspA.

In some polypeptides, SEQ ID NO: 60 separates the first and second monomer of the MspA, SEQ ID NO: 61 separates the second and third monomer of the MspA, SEQ ID NO: 62 separates the third and fourth monomer of the MspA, SEQ ID NO: 63 separates the fourth and fifth monomer of the MspA, SEQ ID NO: 64 separates the fifth and sixth monomer of the MspA, SEQ ID NO: 65 separates the sixth and seventh monomer of the MspA, and SEQ ID NO: 66 separates the seventh and eighth monomer of the MspA.

In some polypeptides, SEQ ID NO: 23 separates the first and second monomer of the MspA, SEQ ID NO: 25 separates the second and third monomer of the MspA, SEQ ID NO:27 separates the third and fourth monomer of the MspA, SEQ ID NO: 29 separates the fourth and fifth monomer of the MspA, SEQ ID NO: 31 separates the fifth and sixth monomer of the MspA, SEQ ID NO: 33 separates the sixth and seventh monomer of the MspA, and SEQ ID NO: 35 separates the seventh and eighth monomer of the MspA.

Some polypeptides further comprises a protease cleavage site positioned between the first amino acid linker and the single-chain MspA and/or a protease cleavage site positioned between the third amino acid linker and the second affinity tag. Some polypeptides comprise one or more first affinity tags, optionally separated by the first amino acid linker. Some polypeptides comprise one or more second affinity tags, optionally separated by the third amino acid linker.

### Systems

Also provided is a system comprising any single-chain Msp polypeptide described herein having a vestibule and a constriction zone that define a tunnel, wherein the tunnel is positioned between a first liquid medium and a second liquid medium, wherein at least one liquid medium comprises an analyte, and wherein the system is operative to detect a property of the analyte. A system can be operative to detect a property of any analyte comprising subjecting an Msp to an electric field such that the analyte interacts with the Msp. A system can be operative to detect a property of the analyte comprising subjecting the Msp to an electric field such that the analyte electrophoretically translocates through the tunnel of the Msp. Also provided is a system comprising an Msp having a vestibule and a constriction zone that define a tunnel, wherein the tunnel is positioned in a lipid bilayer between a first liquid medium and a second liquid medium, and wherein the only point of liquid communication between the first and second liquid media occurs in the tunnel. Moreover, any system described herein can comprise any Msp described herein.

The first and second liquid media can be the same or different, and either one or both can comprise one or more salts, detergents, or buffers. In fact, any liquid media described herein can comprise one or more of a salt, a detergent, or a buffer. Optionally, at least one liquid medium is conductive. Optionally, at least one liquid medium is not conductive. Any liquid medium described herein can comprise a viscosity-altering substance or a velocity-altering substance. The liquid medium can comprise any analyte described herein.

A property of an analyte can be an electrical, chemical, or physical property. An Msp can be comprised in a lipid bilayer in a system or any other embodiment described herein. A system can comprise a plurality of Msps. A system can comprise any Msp described herein. A Msp comprised in a system can comprise a vestibule having a length from about 2 to about 6 nm and a diameter from about 2 to about 6 nm, and a constriction zone having a length from about 0.3 to about 3 nm and a diameter from about 0.3 to about 3 nm, wherein the vestibule and constriction zone together define a tunnel.

### Detection Methods

Further provided is a method for detecting the presence of an analyte, comprising: (a) applying an electric field sufficient to translocate an analyte from a first conductive medium to a second conductive medium in liquid communication through any single-chain Msp(s) described herein (i.e., any plurality of Msps described herein or any plurality of Msps produced by any of the methods described herein) or system comprising a single-chain Msp described herein; and (b) measuring an ion current, wherein a reduction in the ion current indicates the presence of the analyte in the first medium. Optionally, the first and second liquid conductive media are the same. Optionally, the first and second liquid conductive media are different.

In the methods disclosed herein, an Msp can further comprise a molecular motor. The molecular motor can be capable of moving an analyte into or through a tunnel with a translocation velocity or an average translocation velocity that is less than the translocation velocity or average translocation velocity at which the analyte electrophoretically translocates into or through the tunnel in the absence of the molecular motor. Accordingly, in any embodiment herein comprising application of an electric field, the electric field can be sufficient to cause the analyte to electrophoretically translocate through the tunnel. Any liquid medium discussed herein, such as a conductive liquid medium, can comprise an analyte. In the methods comprising measuring an ion current, the analyte interacts with an Msp porin tunnel to provide a current pattern, wherein the appearance of a blockade in the current pattern indicates the presence of the analyte.

The methods disclosed herein can further comprise identifying the analyte. For example, such methods can comprise comparing the current pattern obtained with respect to an unknown analyte to that of a known current pattern obtained using a known analyte under the same conditions. In another example, and not to be limiting, identifying the analyte can comprise (a) measuring the ion current to provide a current pattern, wherein a reduction in the current defines a blockade in the current pattern, and (b) comparing one or more blockades in the current pattern to (i) one or more blockades in the current pattern, or (ii) one or more blockades in a known current pattern obtained using a known analyte.

The analyte can be any analyte described herein. For example, the analyte can be a nucleotide(s), a nucleic acid, an amino acid(s), a peptide, a protein, a polymer, a drug, an ion, a pollutant, a nanoscopic object, or a biological warfare agent. In the methods provided herein, optionally, at least one of the first or second conductive liquid media comprises a plurality of different analytes.

In methods where the analyte is a polymer, for example, a protein, a peptide or a nucleic acid, the method can further comprise identifying one or more units of the polymer. For example, identifying one or more units of the polymer can comprise measuring the ion current to provide a current pattern comprising a blockade for each polymer unit, and comparing one or more blockades in the current pattern to (i) one or more other blockades in the current pattern or (ii) one or more blockades in a current pattern obtained using a polymer having known units. These methods can comprise identifying sequential units of the polymer, for example, and not to be limiting, sequential or consecutive nucleotides in a nucleic acid. In another example, sequential or consecutive amino acids in a polypeptide can be identified using the methods described herein.

The methods provided herein can comprise distinguishing at least a first unit within a polymer from at least a second unit within the polymer. Distinguishing can comprise measuring the ion current produced as the first and second units separately translocate through a tunnel to produce a first and a second current pattern, respectively, where the first and second current patterns differ from each other.

The methods provided herein can further comprise sequencing a polymer. Sequencing can comprise measuring the ion current or optical signals as each unit of the polymer is separately translocated through the tunnel to provide a current pattern that is associated with each unit, and comparing each current pattern to the current pattern of a known unit obtained under the same conditions, such that the polymer is sequenced.

Further provided is a method of sequencing nucleic acids or polypeptides using any of the mutant Msps provided herein. The method comprises creating a lipid bilayer comprising a first and second side, adding a purified Msp to the first side of the lipid bilayer, applying positive voltage to the second side of the lipid bilayer, translocating an experimental nucleic acid or polypeptide sequence through the Msp porin, comparing the experimental blockade current with a blockade current standard, and determining the experimental sequence.

Any of the detection methods provided herein can further comprise determining the concentration, size, molecular weight, shape, or orientation of the analyte, or any combination thereof.

As used herein, a polymer refers to a molecule that comprises two or more linear units (also known as a “mers”), where each unit may be the same or different. Non-limiting examples of polymers include nucleic acids, peptides, and proteins, as well as a variety of hydrocarbon polymers (e.g., polyethylene, polystyrene) and functionalized hydrocarbon polymers, wherein the backbone of the polymer comprises a carbon chain (e.g., polyvinyl chloride, polymethacrylates). Polymers include copolymers, block copolymers, and branched polymers such as star polymers and dendrimers.

Methods of sequencing polymers using Msp are described herein. In addition, sequencing methods can be performed in methods analogous to those described in U.S. Pat. No. 7,189,503, incorporated herein by reference in its entirety. See also U.S. Pat. No. 6,015,714, incorporated herein by reference in its entirety. More than one read can be performed in such sequencing methods to improve accuracy. Methods of analyzing characteristics of polymers (e.g., size, length, concentration, identity) and identifying discrete units (or “mers”) of polymers are discussed in the '503 patent as well, and can be employed with respect to the present Msps. Indeed, an Msp can be employed with respect to any method discussed in the '503 patent.

At present, several types of observable signals can be used as readout mechanisms in nanopore sequencing and analyte detection. An exemplary readout method relies on an ionic blockade current or copassing current, uniquely determined by the identity of a nucleotide or other analyte occupying the narrowest constriction in the pore. This method is referred to as blockade current nanopore sequencing or BCNS. Blockade current detection and characterization of nucleic acids has been demonstrated in both the protein pore ahemolysin (aHL) and solid-state nanopores.

Blockade current detection and characterization has been shown to provide a host of information about the structure of DNA passing through, or held in, a nanopore in various contexts. In general, a blockade is evidenced by a change in ion current that is clearly distinguishable from noise fluctuations and is usually associated with the presence of an analyte molecule at the pore's central opening. The strength of the blockade will depend on the type of analyte that is present. More particularly, a blockade refers to an interval where the ionic current drops below a threshold of about 5-100% of the unblocked current level, remains there for at least 1.0 μs, and returns spontaneously to the unblocked level. For example, the ionic current may drop below a threshold of about, at least about, or at most about 5%, 10%, 15%, 20%, 25%, 30%, 35%, 40%, 45%, 50%, 55%, 60%, 65%, 70%, 75%, 80%, 85%, 90%, 95%, or 100%, or any range derivable therein. Blockades are rejected if the unblocked signal directly preceding or following it has an average current that deviates from the typical unblocked level by more than twice the rms noise of the unblocked signal. Deep blockades are identified as intervals where the ionic current drops <50% of the unblocked level. Intervals where the current remains between 80% and 50% of the unblocked level are identified as partial blockades. Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

As used in the specification and the appended claims, the singular forms “a,” “an,” and “the” include plural referents unless the context clearly dictates otherwise. For example, reference to “a transcript” or “the transcript” may include a plurality of transcripts.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/of” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” Ranges can be expressed herein as from one particular value, and/or to another particular value. When such a range is expressed, also specifically contemplated and considered disclosed is the range from the one particular value and/or to the other particular value unless the context specifically indicates otherwise. It should be understood that all of the individual values and sub-ranges of values contained within an explicitly disclosed range are also specifically contemplated and should be considered disclosed unless the context specifically indicates otherwise. Further, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### Chemicals and Enzymes

Chemicals were of the highest purity available from Sigma Aldrich (St. Louis, MO), Merck (Darmstadt, Germany), Invitrogen (Waltham, MA), or Fisher Scientific (Waltham, MA) unless otherwise noted. The detergent n-octylpolyoxyethylene (OPOE) was from Santa Cruz Biotechnology (Dallas, TX). Restriction enzymes and other molecular biology reagents were from New England Biolabs (Ipswich, MA). Genes were synthesized by GenScript (Piscataway, NJ). The oligonucleotides were obtained from Integrated DNA Technologies (Coralville, IA).

### Bacterial Strains and Growth Conditions

Mycobacterium smegmatis ML712, which lacks the porin genes mspA, mspB, mspC, and mspD (Bezrukov et al., 1993. Probing alamethicin channels with water-soluble polymers. Effect on conductance of channel states. Biophys J. 64(1):16-25), was used for purification of octameric MspA proteins (wtMspA, MspA M1, MspA M2) and grown at 37° C. in 7H9 liquid medium (BD Biosciences) supplemented with 0.2% glycerol and 0.05% Tween 80 or on 7H10 agar (BD Biosciences) supplemented with 0.2% glycerol. Hygromycin was used in concentrations of 50 μg/ml for M. smegmatis ML712. Escherichia coli DH5a was used for cloning experiments and was routinely grown in Luria-Bertani broth (LB) at 37° C. For single-chain MspA production and purification. E. coli Omp8 (Nekolla et al., 1994. Noise analysis of ion current through the open and the sugar-induced closed state of the LamB channel of Escherichia coli outer membrane: evaluation of the sugar binding kinetics to the channel interior. Biophys J. 66(5):1388-13972) was grown in LB broth. Ampicillin or streptomycin were used in concentrations of 100 μg/ml and 50 μg/ml, respectively, for E. coli DH5α and Omp8 growth. See Table 2.

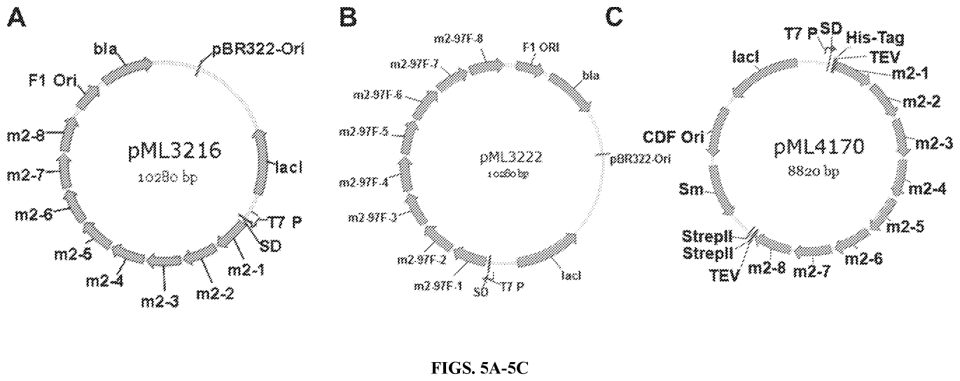

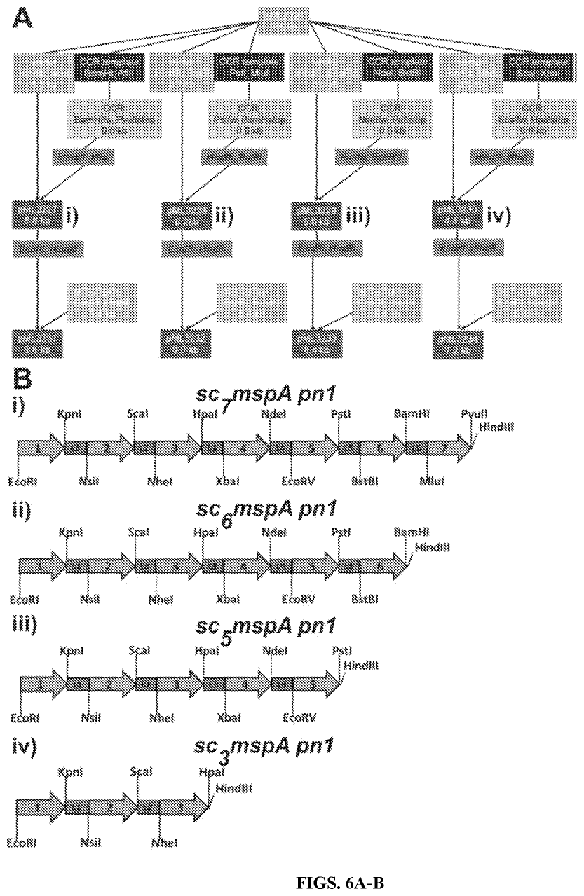

Plasmid Construction Full-length single-chain mspA genes and m2-1 gene with histidine8-tag and last m2-8 with TwinStrepII tag were ordered from GenScript (Piscataway, NJ). The resulting plasmids used in this study (FIG. 5, 6, Table 3) were constructed using standard molecular biology methods. Plasmids were analyzed by digestion with restriction enzymes and by sequencing to verify introduced genes or mutations. Table 3 lists the plasmids used in the Examples. The genes kan, amp, and stm confer resistance to kanamycin, ampicillin, and streptomycin, respectively. Table 4 lists oligonucleotides used, as described in FIG. 6.

### Purification of Octameric MspA Proteins

Purification of MspA M2 was performed as described previously (Bayley et al, Chem. Rev. 100(7): 2575-2594 (2000); Deamer et al., 2016. Three decades of nanopore sequencing. Nat Biotechnol. 34(5):518-524)) with slight modifications. Briefly, Mycobacterium smegmatis ML712 harboring pML844 plasmid was grown for two days at 37° C. Cell pellets were collected, washed and resuspended in OPOE buffer followed by boiling for 30 minutes. The protein extract was precipitated with ice-cold acetone and incubated on ice overnight. The precipitated protein was resuspended in OPOE (0.5% v/v) buffer and applied onto Superdex S200 HiLoad 26/60 column for gel filtration. Fractions of pure MspA M2 were pooled together and used for the experiments described here.

### Expression and Purification of Single-Chain MspA M2 and Single-Chain MspA PN1 Proteins.

E. coli strain Omp8 transformed was used for proteins production and purification. Cells with plasmids encoding different single-chain MspA PN1 constructs were grown overnight at 37° C. in LB medium containing 100 μg/ml ampicillin. Overnight cultures were then diluted into 1 L of fresh LB medium to an OD600 of approximately 0.1 and incubated at 37° C. At OD₆₀₀ of 0.6 expression of the scmspA pn1s was induced with 1.5 mM IPTG. After the induction cells grew for 2 hours at 37° C. followed by inclusion bodies purification as described elsewhere (Kasianowicz et al., 1996. Characterization of individual polynucleotide molecules using a membrane channel. Proc Natl Acad Sci USA. 93(24):13770-13773). Briefly, after sonication cells were centrifuged at 1,500 g for 10 min at 4° C. to remove cell debris. Triton X100 (1%, v/v, final) was added to solubilize membrane proteins and the mixture was incubated on ice for 10 minutes. Then the sample was centrifuged at 7,000 g for 20 min at 4° C. to collect insoluble pellet. The pellet was washed three more times with lysis buffer to remove Triton X100. The resulting pellet containing inclusion bodies was resuspended in 8 M urea and incubated overnight at room temperature. Next, inclusion bodies were separated on 8% polyacrylamide gel followed by staining with Simply Blue Safe Stain (Invitrogen). The single-chain MspA PN1 proteins were then eluted from the gel as follows. Band corresponding to theoretical molecular weight of single-chain construct was excised from a gel with a clean razor. The gel bands were crushed and protein elution buffer (25 mM HEPES, 150 mM NaCl, 0.5% (v/v) OPOE, pH 7.5) was added followed by brief sonication on ice to further disperse gel particles. The ratio of gel volume to protein elution buffer was 1:3. The mixture was placed on a rotary shaker and incubated overnight at 30° C. After incubation the sample was centrifuged at 16,000 g for 5 minute to pellet polyacrylamide gel pieces. The supernatant contained eluted protein which was used for refolding by dialysis against 2 L of refolding buffer (buffer (150 mM NaCl, 50 mM NaPi, 200 mM L-Arginine, 800 mM urea, 0.5% OPOE, 1 mM PMSF, Complete Protease Inhibitor cocktail, 0.02% sodium azide, pH 8.0). Concentration of the refolded protein was measured by BCA kit (Thermo). The refolded protein was used immediately or frozen at −20° C. with glycerol (50%, v/v) for storage.

### Expression and Purification of Single-Chain Double-Tagged MspA M2

E. coli strain Omp8 transformed with pML4170 was grown overnight at 37° C. in LB medium containing 50 μg/ml streptomycin. The cells were then diluted into 1-2 L of fresh medium to give OD₆₀₀ of 0.1. When OD₆₀₀ reached 0.6 the cells were induced with 1 mM IPTG (final). The cultures were then transferred to 18° C. and grown for 14 hours. Harvested cells were washed in PBS and resuspended in lysis buffer in the ratio of 1:5 (150 mM NaCl, 50 mM NaPi, pH 7.4 supplemented with Benzonase (Novagen) and Complete Protease Inhibitor cocktail (Roche). The cells were sonicated on Misonix sonicator for 20 minutes on ice (30 s on/off cycle, 50 watts). The lysate was then used for inclusion bodies purification as described in the previous paragraph. Inclusion bodies in 8 M urea were loaded on NiNTA agarose resin (Qiagen) to bind single-chain MspA. Ni-affinity purification was performed in denaturing conditions with buffer composition of 150 mM NaCl, 50 mM NaPi, 6 M urea, pH 7.4. scMspA was eluted with denaturing buffer containing 700 mM imidazole. Elution fractions were pooled, concentrated on Amicon spin column (Millipore) with 50 kDa cutoff, and loaded onto Superdex S200 26/60 HiLoad column (GE Life Sciences) for gel filtration in denaturing consitions (150 mM NaCl, 50 mM NaPi, 6 M urea, 0.2% (w/v) SDS, pH 7.5). Fractions containing scMspA were combined, concentrated and used for StrepII tag affinity purification on Strep-Tactin XT resin (IBA). 50 mM biotin in 150 mM NaCl, 50 mM NaPi, 6 M urea, pH 8.0 buffer was used to elute scMspA protein. The elution fractions were combined and OPOE (0.5%, v/v, final) was added prior to refolding by dialysis overnight at room temperature against 2 L of refolding buffer (150 mM NaCl, 50 mM NaPi, 200 mM L-Arginine, 800 mM urea, 0.5% OPOE, 1 mM PMSF, Complete Protease Inhibitor cocktail, 0.02% sodium azide, pH 8.0). This sample is referred to as refolded sample. As the final step to remove contaminants and refolding buffer components refolded sample was concentrated on Amicon spin column (Millipore) with 50 kDa cutoff and loaded on Supredex S200 Increase 10/300 GL column (GE Healthcare) and eluted with 150 mM NaCl, 50 mM NaPi, 0.5% OPOE, pH 7.4 buffer. Individual fractions were used for lipid bilayer experiments and downstream analysis.

### Denaturing of Single-Chain MspA

For all experiments equal amounts of protein (2 μg) were used. Octameric MspA M2 purified from Mycobacterium smegmatis ML712 or refolded single-chain MspA proteins were mixed with DMSO (80% v/v, final). The mixture was incubated for 15 min at 99° C. followed by addition of 10 volumes of ice-cold acetone to precipitate the protein and incubated on ice for 15 min. Precipitated samples were centrifuged at 16,000 g for 15 min at 4° C. The protein pellet was dried under the vacuum to remove acetone. After drying the samples were resuspended in the initial volume, mixed with loading dye and separated on polyacrylamide gel.

### Lipid Bilayer Measurements

Lipid bilayer experiments were performed in a custom made lipid bilayer apparatus as previously described (Deamer et al., 2016). Briefly, a Teflon cuvette with 10 ml volume is separated into to compartments (cis- and trans-) by a wall with an aperture of approximately 1 mm in diameter. Ag/AgCl electrodes were bathed in a 1 M KCl, 10 mM HEPES, pH 7.4 electrolyte solution. The cuvette was prime on both side of the aperture with 2% diphytanoylphosphatidylcholine (DPhPC; Avanti Polar Lipids) in chloroform. Lipid membranes were painted across aperture from a solution of 1% of DPhPC in n-decane. The samples were added to the both sides of the cuvette. Baseline and detergent-containing buffers were examined to exclude contamination and detergent interference. Single channel conductances for more than 100 pores for a protein sample were recorded. Recording were performed at −10 mV potential. Current was recorded using Keithley 428 Current Amplifier with a filter rise time of 30 ms, and digitized by a computer equipped with Keithley Metrabyte STA 1800 U interface. The data were recorded with Test Point 4.0 software (Keithley). The raw data were analyzed using IGOR Pro 5.03 (WaveMetrics) using a macro provided by Dr. Harald Engelhardt. The data were further analyzed in SigmaPlot 11.0 (Systat Software) to generate graphs shown here.

### PBD-PEO Polymer Membrane Formation and Single-Channel Nanopore Measurements

The chip with 100 μm SU-8 wedge-on-pillar aperture (Niederweis et al., Mol. Microbiol. 33(5): 933-945 (1999)) was glued and sealed to a custom designed fluidic cell, separating cis and trans chambers. The aperture was pretreated by 4 mg/ml poly(1,2-butadiene)-b-poly(ethylene oxide) (PBD₁₁-PEO₈) block-copolymer (Polymer Source) dissolved in hexane. After hexane solvent evaporated, a dry layer of polymer was formed on the aperture edge. The cis and trans chamber were filled with buffer, with insertion of a pair of Ag/AgCl electrolytes which connected to an Axon 200B patch-clamp amplifier. The polymer membrane was painted across the pretreated aperture using 8 mg/ml polymer dissolved in decane. More than 60 mins waiting time is needed for the polymer membrane to thin down until it forms a bilayer (membrane capacitance range: 60-80 pF). 0.063 nM of octameric MspA M2 or 4.2 nM of sc₈MspA_{dt} M2 was added to the cis chamber to observe a single pore insertion. DNA hairpins were added to cis chamber. The data were collected at 250 kHz sampling rate with 10 kHz low pass filter applied. All oligonucleotides were purchased from Integrated DNA Technologies (IDT).

### Results

### Purification and Characterization of Single-Chain MspA.

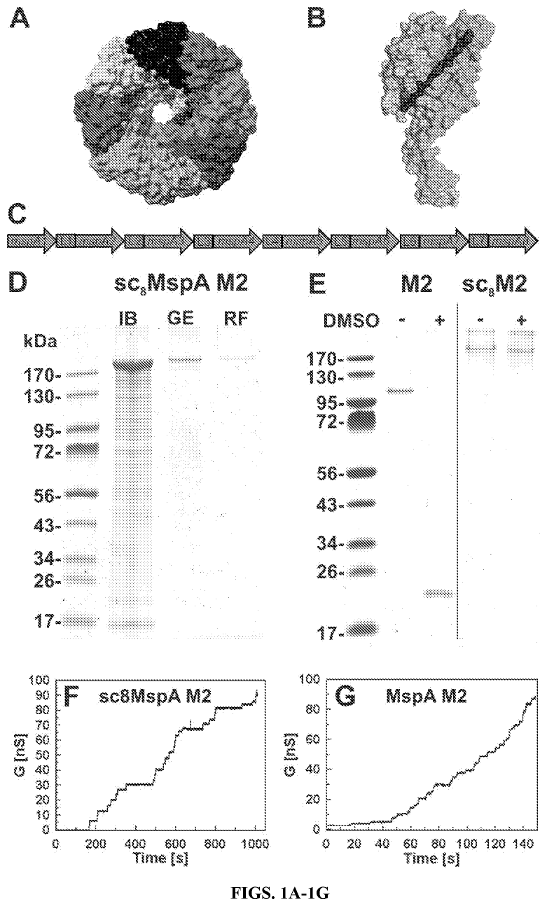

Eight MspA subunits assemble in the outer membrane of Mycobacterium smegmatis to produce a central water-filled channel (FIG. 1A). The C-terminus of a monomer can be connected to the N-terminus of a neighboring subunit by a 17-mer peptide linker due to the close proximity of the termini (Pavlenok et al., PloS One 7(6): e38726 (2012)). Here, the same approach was used to synthesize a single-chain mspA m2 (scmspA m2) gene where eight mspA m2 genes encoding the mutations D90N/D91N/D93N/D118R/D134R/E139K, which enable efficient DNA capture and translocation (Butler et al., Proc. Natl. Acad. Sci USA 105(52): 20647-20652 (2008)), are connected by DNA fragments encoding (GGGGS)₃ (SEQ ID NO: 22) or (GGGGS)_{X} (SEQ ID NO: 19), wherein X is an integer, peptide linkers (FIG. 1B; Table 1). Each gene is flanked by unique restriction sites to enable specific modifications of each MspA subunit (Table 1). Efforts were first made to produce scMspA M2 protein in the porin quadruple mutant M. smegmatis ML712, which lacks all four msp genes (Pavlenok et al., FEMS Microbiol. Lett. 363(7) (2016)). However, all scmspA m2 genes in clones producing MspA protein were scrambled and protein quantities were very low. Then, E. coli was used to produce scMspA M2 protein. To reduce the probability of homologous recombination between the individual mspA genes, every second codon in the mspA subunit genes 2 through 8 was altered to generate DNA sequence differences without altering the amino acid sequence. In addition, the scmspA m2 DNA sequence was altered for optimal expression in E. coli and the signal peptide was removed for cytoplasmic expression of MspA. The synthetic scmspA m2 gene was expressed under the control of the T7 promoter using the plasmid pML3216 (FIG. 5) in the BL21(DE3)omp8 strain lacking the three major porins of E. coli (Prilipov et al., FEMS Microbiol. Lett. 163(1): 65-72 (1998)) to avoid contamination with endogenous porins. MspA protein (166 kDa theoretical mass) was purified from inclusion bodies by gel electrophoresis and excision of a protein band of 170 kDa and refolded in a buffer containing the detergent octyl polyoxyethylene (OPOE) (FIG. 1D). The protein yield was 12 μg of purified sc₈MspA M2 per single preparation from four polyacrylamide gels (approximately 190 μg per liter of E. coli culture).

TABLE 1
Sequences of linkers that connect mspA genes in scmspA constructs.

| Linker | Sequences: amino acid (N--C-term); nucleotides (5′-3′) | SEQ ID NO: |
|---|---|---|
| L1 | KpnI NsiI | |
| | G T G G G G S G G G G S G G G G S M H | 23 |
| | GGT ACC GGC GGT GGC GGT AGT GGC GGT GGC GGT TCC GGC GGT GGC GGT TCA ATG CAT | 24 |
| L2 | ScaI NheI | |
| | S T G G G G S G G G G S G G G G S A S | 25 |
| | AGT ACT GGC GGT GGC GGT TCG GGC GGT GGC GGT AGC GGC GGT GGC GGT AGC GCT AGC | 26 |
| L3 | HpaI XbaI | |
| | V N G G G G S G G G G S G G G G S S R | 27 |
| | GTT AAC GGC GGT GGC GGT TCT GGC GGT GGC GGT AGT GGC GGT GGC GGT AGC TCT AGA | 28 |
| L4 | NdeI EcoRV | |
| | H M G G G G S G G G G S G G G G S D I | 29 |
| | CAT ATG GGC GGT GGC GGT TCC GGC GGT GGC GGT TCA GGC GGT GGC GGT TCG GAT ATC | 30 |
| L5 | PstI BstBI | |
| | L Q G G G G S G G G G S G G G G S F E | 31 |
| | CTG CAG GGC GGT GGC GGT AGC GGC GGT GGC GGT TCT GGC GGT GGC GGT AGT TTC GAA | 32 |
| L6 | BamHI MluI | |
| | G S G G G G S G G G G S G G G G S T R | 33 |
| | GGA TCC GGC GGT GGC GGT TCA GGC GGT GGC GGT TCG GGC GGT GGC GGT AGC ACG CGT | 34 |
| L7 | PvuII Afl II | |
| | Q L G G G G S G G G G S G G G G S L K | 35 |
| | CAG CTG GGC GGT GGC GGT TCT GGC GGT GGC GGT AGT GGC GGT GGC GGT TCC CTT AAG | 36 |

TABLE 2
Strains

| | | Source or |
|---|---|---|
| Strain | Parent strain and relevant genotype | reference |
| E. coli DH5α | recA1; endA1; gyrA96; thi; relA1; hsdR17(r_{K}⁻, m_{K}⁺); supE44; | (7) |
| | ϕ80ΔlacZΔM15; ΔlacZ(YA-argF)UE169 | |
| E. coli | BL21(DE3), ΔlamB, ompF::Tn5, ΔompA, ΔompC | (2) |
| BL21(DE3) | | |
| Omp8 | | |
| M. smegmatis | ΔmspA::FRT, ΔmspB::loxP, ΔmspC::FRT, ΔmspD::FRT, attB | (1) |
| ML712 | L5::FRT-pNIT-cpnT_{NTD}; Sm^{R} | |

TABLE 3
Plasmids

| | | Source or |
|---|---|---|
| Plasmid | Components and properties | reference |
| pET21a+ | | Novagen |
| pML3215 | pUC57 derivative; scmspA m2, kan | this study |
| pML3216 | pET21a + derivative;; scmspA m2, amp | this study |
| pML3221 | pUC57 derivative; scmspA pn1, kan | this study |
| pML3222 | pET21a + derivative; scmspA m2, amp | this study |
| pML3227 | pML3221 derivative; sc7mspA pn1, kan | this study |
| pML3228 | pML3221 derivative; sc6mspA pn1, kan | this study |
| pML3229 | pML3221 derivative; sc5mspA pn1, kan | this study |
| pML3230 | pML3221 derivative; sc3mspA pn1, kan | this study |
| pML3231 | pET21a + derivative; sc7mspA pn, amp | this study |
| pML3232 | pET21a + derivative; sc6mspA pn, amp | this study |
| pML3233 | pET21a + derivative; sc5mspA pn, amp | this study |
| pML3234 | pET21a + derivative; sc3mspA pn, amp | this study |
| pML4170 | pCDFDuet-1 backbone, sc8mspAdt m2, stm | this study |
| pML844 | pMN016 derivative; mspA | (8) |
| | D90N/D91N/D93N/D118R/D134R/E139K | |
| | (m2mspA) | |
| pML844F | pML844 derivative; MspA PN1 (M2 + P97F) | this study |

TABLE 4
Oligonucleotides

| Oligonucleotide | Sequence (5′→3′) | SEQ ID NO: | Notes |
|---|---|---|---|
| PMSSEQ1 | CGTTCTCGGCTCGATGATCC | 37 | Cloning of mspA |
| COLE1 | GCGAGTCAGTGAGCGAGGAAGCG | 38 | Cloning of mspA |
| P97F fw | GGTAACATCACCGCTTTCCCGTTCGGC | 39 | Cloning of mspA |
| BamHI fw | TGAATGGATCCGGCG | 40 | Cloning of mspA |
| PvulIstop | TTTTTTAAGCTTGTCACCCAGCTGGTT | 41 | Cloning of mspA |
| PstIfw | GCTCGCTGCAGGGCC | 42 | Cloning of mspA |
| BamHstop | TTTTTTAAGCTTTCAGCCGGATCCATT | 43 | Cloning of mspA |
| NdeIfw | AACCATATGGGCGGT | 44 | Cloning of mspA |
| PstIstop | TTTTTTAAGCTTTCAGCCCTGCAGGTT | 45 | Cloning of mspA |
| ScaIfw | TGAACAGTACTGGCG | 46 | Cloning of mspA |
| HpaIstop | TTTTTTAAGCTTTCAGCCGTTAACGTTC | 47 | Cloning of mspA |
| Scalfw | TGAACAGTACTGGCG | 48 | Cloning of mspA |
| hp14-dT50 | GCTGGCTCTGTTGCTCTCTCGCAACAGAG | 49 | DNA hairpins with T50 |
| | CCAGCTTTTTTTTTTTTTTTTTTTTTTTTTTT | | tail |
| | TTTTTTTTTTTTTTTTTTTTTTT | | |
| hp14-dA50 | GCTGGCTCTGTTGCTCTCTCGCAACAGAG | 50 | DNA hairpins with A50 |
| | CCAGCAAAAAAAAAAAAAAAAAAAAAAAAA | | tail |
| | AAAAAAAAAAAAAAAAAAAAAAAAA | | |
| hp14-dC27 | GCTGGCTCTGTTGCTCTCTCGCAACAGAG | 51 | DNA hairpins with C27 |
| | CCAGCCCCCCCCCCCCCCCCCCCCCCCC | | tail |
| | CCC | | |
| hp14-dG3dA47 | GCTGGCTCTGTTGCTCTCTCGCAACAGAG | 52 | DNA hairpins with |
| | CCAGCGGGAAAAAAAAAAAAAAAAAAAAA | | G3A47 tail |
| | AAAAAAAAAAAAAAAAAAAAAAAAAA | | |

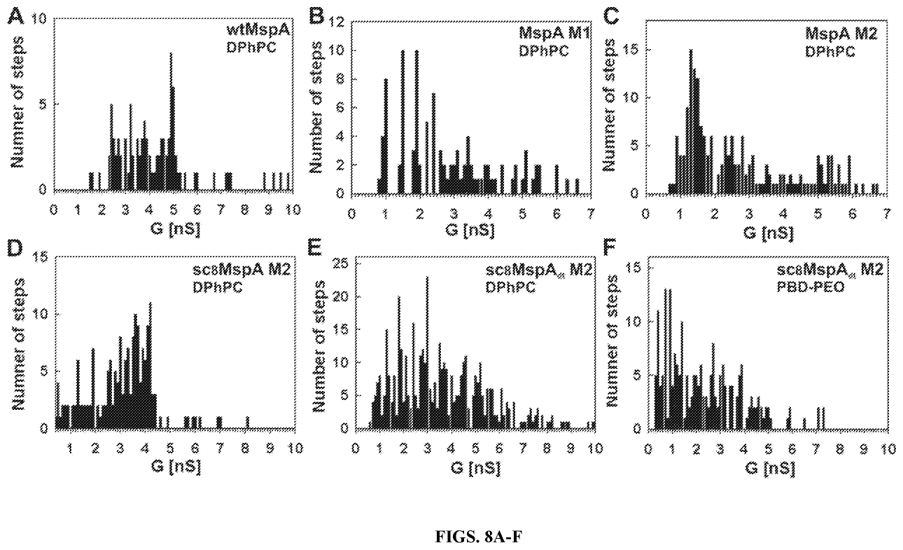

Octameric MspA is an extremely stable protein that does not denature after boiling for 10 min in 2% SDS and other harsh denaturing conditions. To dissociate octameric MspA into its subunits, the purified proteins were boiled in 80% (v/v) dimethylsulfoxide (DMSO) (25). Only octameric MspA M2 dissociated into monomers, while sc₈MspA M2 was stable demonstrating that all eight subunits are covalently linked and that full-length scMspA was purified (FIG. 1E). To examine whether sc₈MspA M2 forms functional channels, lipid bilayer experiments were performed in diphytanoyl phosphatidylcholine (DPhPC) membranes of 1 mm in diameter in a Montal-Mueller setup (25). Addition of both octameric MspA M2 purified from M. smegmatis (FIG. 1G) and recombinant sc₈MspA M2 protein to the membranes resulted in a step-wise current increase indicative of channel insertions (FIG. 1F), while no channel activity was observed when only detergent-containing buffer was added. Analysis of the single channel insertions revealed similar conductance values ranging from 0.5 to 6 nS for octameric MspA M2 and from 0.2 to 4.5 nS for single chain MspA (FIG. 8).

These wide ranges of single channel conductances were also observed for other octameric MspA proteins such as wt MspA and MspA M1 (FIG. 8) and seem to be an intrinsic feature of the MspA pore. Taken together, these experiments demonstrate that sc₈MspA M2 forms functional pores and show that it is feasible to convert an oligomeric pore protein into a functional monomeric protein enabling asymmetric mutations to improve channel properties.

Single-Chain MspA Variants with Altered Subunit Stoichiometries Form Functional Channels.

To highlight the advantages of scMspA in tailoring the pore for specific applications, the channel diameter was changed as one of the most important properties determining the interactions of the pore constriction with the analyte. Previously, the constriction diameter could only be altered by mutations of individual amino acids in the protein monomer, which also changes the chemical nature of the interactions with the analyte. A monomeric pore consisting of identical subunits enables for the first time to vary the channel diameter by altering the subunit stoichiometry. To this end, scMspA variants were designed with three, five, six, seven, and eight subunits based on MspA M2 with an additional P97F mutation (Table 5).

TABLE 5
Single-chain MspAs

| Single-chain | |
|---|---|
| MspA | |
| protein | Protein description |
| sc₈MspA M2 | Eight covalently linked MspA M2 subunits; MspA M2 |
| | subunit: D90N/D91N/D93N/D118R/D134R/E139K |
| sc₈MspA_{dt} M2 | Eight covalently linked MspA M2 subunits flanked by |
| | histidine₈-tag (N-terminus) and Twin-Strepll tag (C- |
| | terminus) |
| sc₈MspA PN1 | Eight covalently linked MspA PN1 subunits; MspA PN1 |
| | subunit: D90N/D91N/D93N/P97F/D118R/D134R/E139K |
| sc₇MspA PN1 | Seven covalently linked MspA PN1 subunits. |
| sc₆MspA PN1 | Six covalently linked MspA PN1 subunits. |
| sc₅MspA PN1 | Five covalently linked MspA PN1 subunits. |
| sc₃MspA PN1 | Three covalently linked MspA PN1 subunits. |

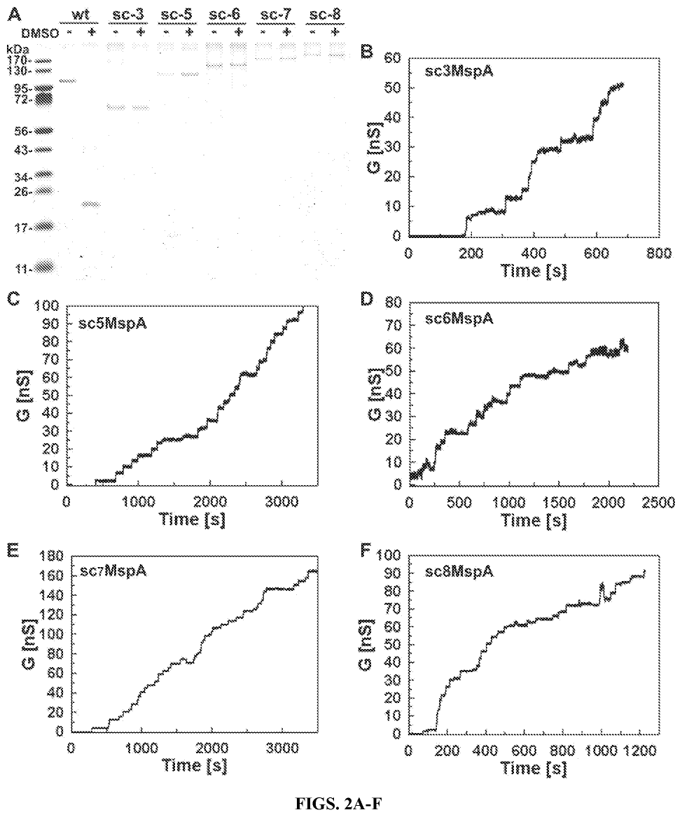

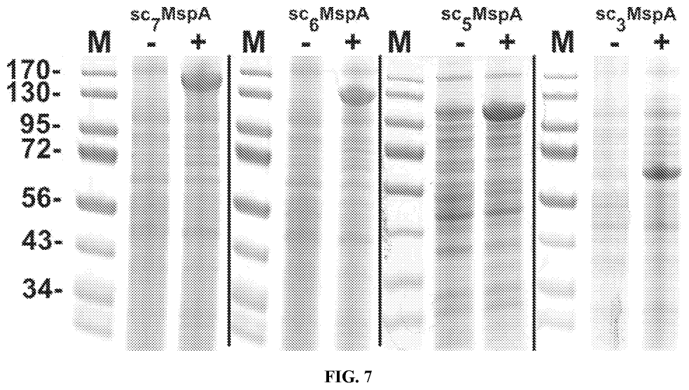

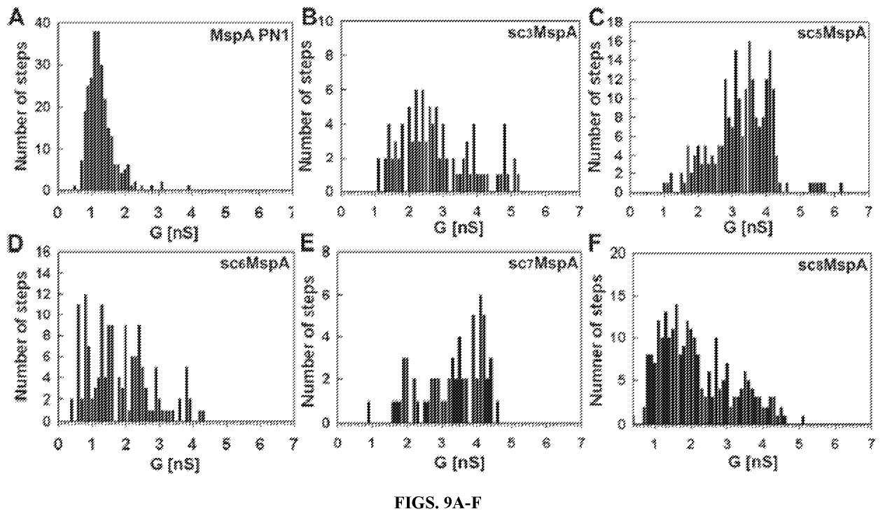

This additional mutation was chosen because octameric MspA PN1 has a well-defined channel distribution with a peak at 1.2 nS (FIG. 9A) in contrast to all other examined octameric MspA pores (FIG. 8). The corresponding proteins were named scₓMspA PN1 with “x” numbers of covalently linked subunits and were purified from E. coli BL21(DE3)omp8 as described above. The target proteins accounted for approximately 10% of proteins in the whole-cell lysate as determined by quantitative image analysis of protein gels (FIG. 7). The apparent molecular masses for sc₃MspA PN1, sc₅MspA PN1, sc₆MspA PN1, and sc₇MspA PN1 proteins were 60 kDa, 110 kDa, 120 kDa, and 140 kDa, respectively (FIG. 2A), and were in a good agreement with the theoretical molecular masses. To examine whether the scMspA PN1 subunits are covalently linked, the proteins were denatured by boiling in 80% (v/v) DMSO. DMSO treatment dissociated octameric MspA M2 protein into its 20 kDa monomers, while all scMspA PN1 proteins were stable under these conditions (FIG. 2A) demonstrating that the subunits of these proteins are indeed covalently linked. To examine the channel-forming properties of scMspA PN1 pores, planar lipid bilayer experiments were performed as previously described (29). In these experiments addition of 70-840 ng of refolded proteins to the membrane resulted in a step-wise current increase indicative of channel insertions into lipid membranes (FIG. 2B-F). The channel insertion frequencies were similar for all scMspA PN1 proteins. The scMspA PN1 pores showed conductance values ranging between 0.5 to 5 nS (FIG. 9) similar to sc₈MspA M2 and octameric MspA proteins (FIG. 8). Interestingly, the distribution peaks shifted to larger single channel conductances for the sc₃MspA PN1, sc₅MspA PN1 and sc₇MspA PN1 variants (FIG. 9), indicating the presence of pores with more than eight subunits. Taken together, these experiments demonstrated that it is feasible to construct MspA pores with altered stoichiometries using covalently linked subunits. Thus, the single-chain concept enables the design of MspA pores with constriction zone diameters optimized for translocating different substrates, e.g. in nucleic acid and polypeptide sequencing.

### Improvement of the Purification of Recombinant Single-Chain MspA Protein.

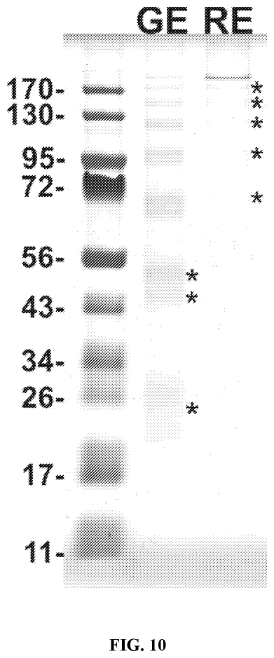

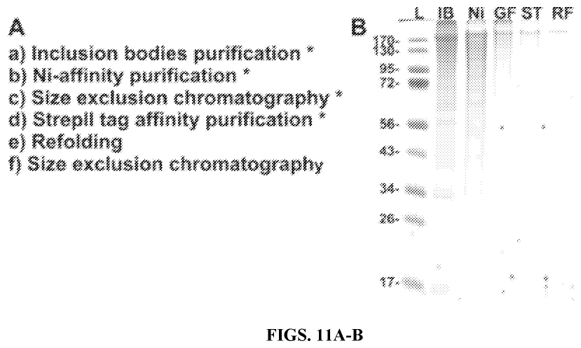

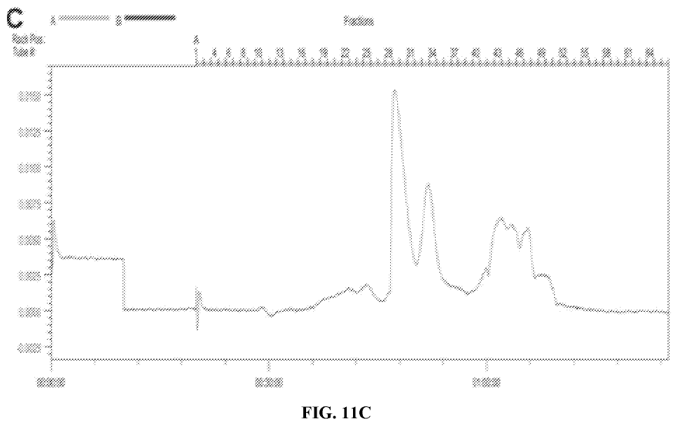

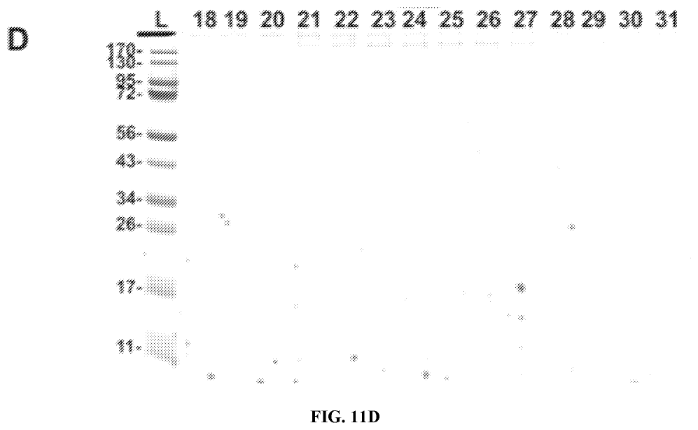

While the above experiments demonstrated that single-chain MspA produces functional pores, the purification based on gel extraction is labor intensive and inefficient resulting in low yields of approximately 10 μg per preparation. ˜98% of the initial amount of scMspA proteins in the inclusion bodies were lost during this process. One of the main challenges was to separate full-length sc₈MspA M2 from its many degradation products which are only marginally smaller, i.e. these degradation products may have cleaved linker peptides which appear to be very susceptible to proteolysis (FIG. 10). To address this issue, the fact that both termini of MspA are accessible on the outside of the MspA channel was exploited (FIG. 1). Thus, a Hiss-tag was added at the N-terminus and a Twin-Strep II tag at the C-terminus flanking the sc₈mspA m2 gene (FIG. 3A). This protein is referred to as double-tagged single-chain MspA M2 (sc₈MspA_{dt} M2). The plasmid pML4170 carrying sc₈mspA_{dt} m2 was transformed into E. coli BL21(DE3)Omp8 for protein production and purification. Sc₈MspA_{dt} M2 was purified under denaturing conditions using subsequent Ni(II)- and streptavidin affinity chromatography to isolate full-length protein (FIGS. 3B,C, 11). Refolding in the detergent OPOE and a final size exclusion (FIG. 11) yielded 0.2 mg per liter of culture per single preparation of apparently pure protein (FIG. 3 B, C). Importantly, no contaminating degradation products were observed (FIG. 10) in the refolded protein sample as evident from western blot on FIG. 3C. As a quality control, denaturing experiments were performed with 80% DMSO. Only octameric MspA M2 dissociated into its monomeric subunits of 20 kDa while sc₈MspA_{dt} M2 was stable indicating that all eight subunits are covalently linked in the purified refolded protein (FIG. 3D). Overall, the combination of N- and C-terminal affinity tags enabled purification of full-length scMspA protein with a 20-fold increase in protein yield per single preparation in comparison to the previous gel excision method.

### Channel-Forming Properties of Single-Chain MspA in Large Membranes.

To examine whether sc₈MspA_{dt} M2 forms functional channels, lipid bilayer experiments were performed in a Montal-Mueller setup using cuvettes with an aperture diameter of 1 mm. As expected, refolded sc₈MspA_{dt} M2 had channel-forming activity in DPhPC membranes as shown by the stepwise current increase after addition of the protein (FIG. 3E). Analysis of 432 insertions from 8 different membranes showed predominant conductance peaks of 1.3 nS, 1.9 ns, and 3.0 nS (FIG. 8E). Notably, sc₈MspA_{dt} M2 had a broader conductance distribution than sc₈MspA PN1 (FIG. 9F). These differences may be the result of different purification methods, the absence of the P97F mutation near the constriction zone of sc₈MspA_{dt} M2 compared to sc₈MspA PN1, and/or the presence of purification tags on the termini of sc₈MspA_{dt} M2.

Octameric MspA M2 forms channels in bilayers composed of poly(1,2-butadiene)-b-poly(ethylene oxide) (PBD-PEO) polymer (10). PBD-PEO bilayers have a two- to three-fold increased lifetime and are more robust towards chemicals and high voltages than membranes made from biological lipids, and can be used in nanopore sequencing experiments (10). Thus, the channel properties of scMspA insertions were also examined in PBD-PEO bilayers. Similarly to DPhPC bilayers, insertions of sc₈MspA_{dt} M2 into PBD-PEO bilayers (FIG. 3F) were observed. Analysis of 207 insertions from 28 membranes showed broad distribution of single-channel conductances (FIG. 8F) in PBD-PEO bilayers similar to those obtained for sc₈MspA_{dt} M2 in DPhPC bilayers. It should be noted that insertion frequency of sc₈MspA_{dt} M2 was reduced in PBD-PEO bilayers in comparison to DPhPC membranes. Taken together, these experiments showed that sc₈MspA_{dt} M2 forms functional channels in both DPhPC and PBD-PEO bilayers.

### Hairpin Translocation Through the Single-Chain MspA Pore.

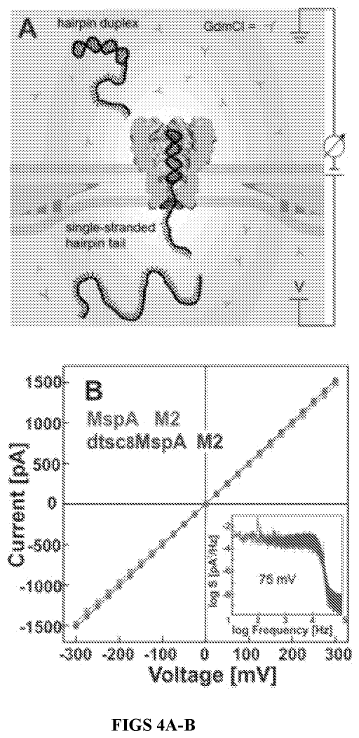

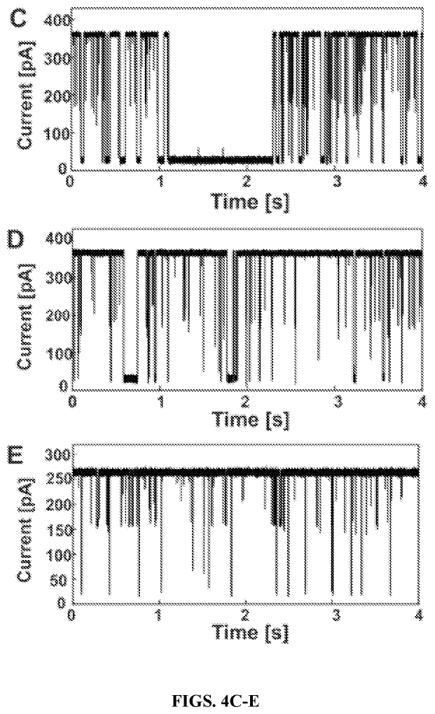

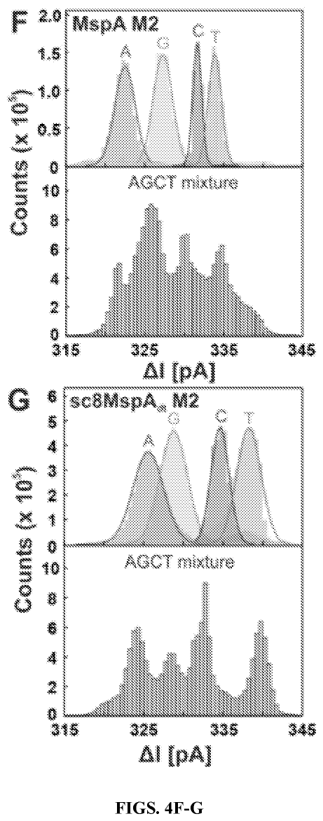

To examine whether the nucleotide recognition capability of MspA is preserved in scMspA, sc₈MspA_{dt} M2 in DNA hairpins experiments were performed. This assay is based on distinct residual currents when the single-stranded homopolymer tail is located inside the constriction zone (Table 4), while the double-stranded region of the DNA hairpins is stalled in the lumen of the MspA pore and temporarily prevents translocation (FIG. 4A). Single sc₈MspA_{dt} M2 pores were inserted into poly(1,2-butadiene)-b-poly(ethylene oxide) (PBD₁₁-PEO₈) block-copolymer bilayer membranes with a diameter of 100 μm and bathed in a buffer containing 10 mM Tris (pH 7.5), 1 M KCl and 2 M guanidinium chloride (GdmCl). Based on previous data (10), GdmCl was used in the electrolyte solution because it induces a semi-melting state of DNA hairpins enabling smooth DNA passage through the nanopore at low applied voltage resulting in increased DNA capture rate and decreased trapping time.

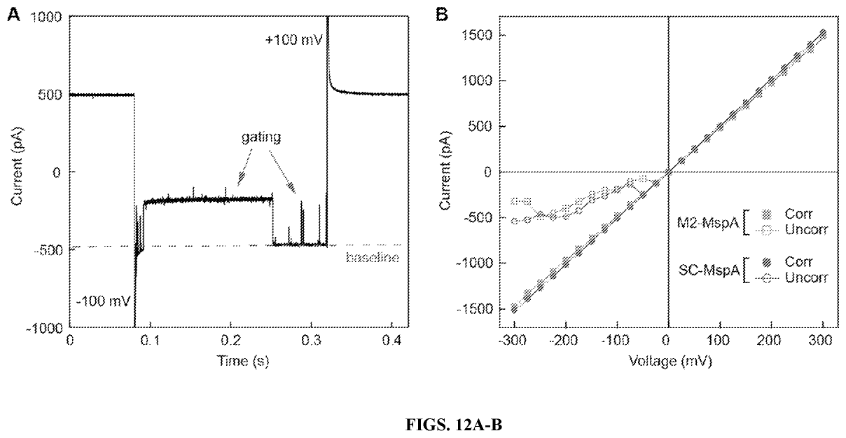

The current-voltage curves and power spectral density plots reveal that the conductance and trace noise are almost identical for sc₈MspA_{dt} M2 and octameric MspA M2 (FIG. 4B). Both proteins exhibited gating when negative voltage was applied (FIG. 12A). Since the gating conductance varies for the same MspA pore even at the same voltage, the ungated current values were used for the IV curves resulting in identical symmetric IV curves for both sc₈MspA_{dt} M2 M2 and MspA M2 (FIG. 4B). Uncorrected IV curves for the same raw data are shown in FIG. 12B. Interestingly, the protein concentration required to observe channel insertions was approximately 60-fold higher for sc₈MspA_{dt} M2 (4.2 nM) than for octameric MspA M2 (0.063 nM). Consistent with previous results in large aperture bilayers (FIG. 8F), a wide conductance distribution was observed for individual sc₈MspA_{dt} M2 channel ranging from 1 nS to 5.5 nS. However, only the pore with conductance of around 4.9 nS resulted in clean and deep DNA translocation signals. This appears to be a general property of the MspA pore as populations of pores with different conductance levels were previously observed in hairpin DNA experiments with octameric MspA isolated from M. smegmatis (Pavlenok et al., 2012).

The current traces after addition of the poly-dT hairpin to sc₈MspA_{dt} M2 and MspA M2 show current blockades resulting from translocation of the DNA hairpin through the pore in GdmCl-containing buffer (FIG. 4C, D). Similar current blockades were observed with the poly-dT hairpin and sc₈MspA_{dt} M2 when no GdmCl was present in the electrolyte buffer (FIG. 4E). However, a higher voltage of 140 mV instead of 75 mV was necessary to overcome the energy barrier for DNA translocation in accordance with previous hairpin experiments with octameric MspA where 140 to 180 mV was used for hairpin DNA translocation (12). These data further indicate that GdmCl improves both capture and translocation of hairpin DNA through the pore.

### Nucleotide Recognition by Single-Chain MspA.

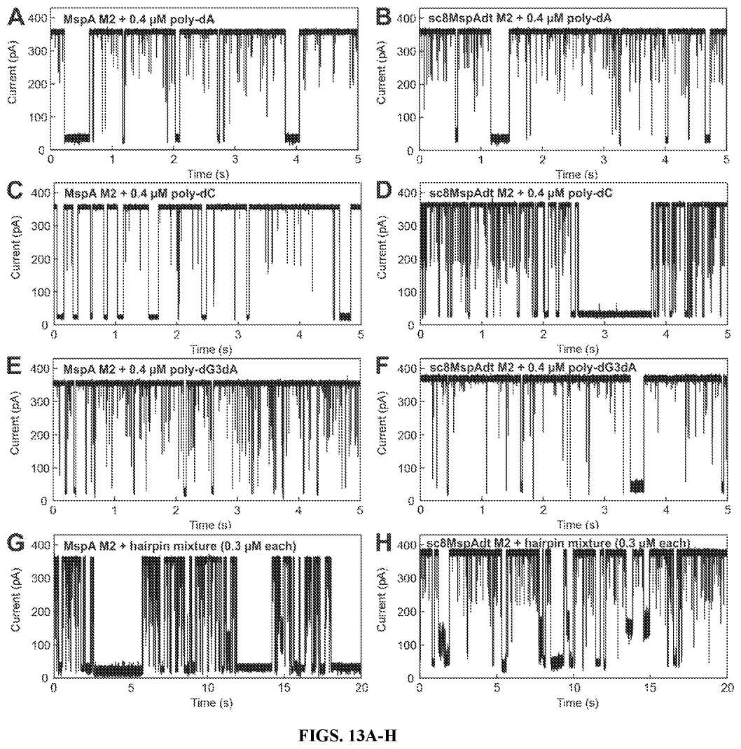

To examine nucleotide recognition by sc₈MspA_{dt} M2, DNA hairpins were used with a duplex region of 14 nucleotides and a homopolymer tail of 50 nt (hp14-dT50 (poly-dT), hp14-dA50 (poly-dA), hp14-dC27 (poly-dC), and hp14-dG3dA47 (poly-dG) (Table 3, FIG. 13). It should be noted that only three dG nucleotides were used in the “poly”dG hairpin tail in otherwise dA background to avoid G-tetrad formation as in previous experiments (12). Histograms of the current change ΔI (current I in the presence of a hairpin minus the baseline current level I₀) are shown for the four individual DNA hairpins and mixture of four hairpins with octameric MspA M2 (FIG. 4F) and sc₈MspA_{dt} M2 (FIG. 4G), respectively. Fitted Gaussians for each individual hairpin with a homopolymer tail were well-resolved and separated, and looked almost identical for both MspA M2 and sc₈MspA_{dt} M2 (FIG. 4F, 4G, top panel). The partial overlap between purines was more pronounced for sc₈MspA_{dt} M2, nonetheless, individual peaks for poly-dA and poly-dG were still distinguished (FIG. 4G, top panels). Remarkably, the histograms of DNA hairpin mixture clearly show four separate peaks representing the four different nucleotides, which match the current blockades for individual hairpin peaks (FIG. 4F, 4G, bottom panels). In this experiment sc₈MspA_{dt} M2 had better resolving properties than octameric MspA M2. In conclusion, these DNA hairpin experiments showed that sc₈MspA_{dt} M2 and octameric MspA M2 have very similar capabilities in distinguishing all four DNA nucleotides. These results indicate that covalently linking of all eight MspA subunits preserves the nucleotide recognition properties of the MspA pore and open many avenues to tailor the channel properties of the MspA pore for specific sensing applications.

### Properties and Potential Applications of Single-Chain MspA Nanopores

By using the exemplary constructs and purification methods disclosed herein, single-chain MspA can be produced as a full-length protein with many asymmetric mutations opening numerous avenues to improve the performance of MspA in DNA sequencing. (i) Distinct amino acids in the MspA constriction zone will enable different chemical interactions with the DNA nucleobases. Asymmetric mutations open numerous avenues to increase the specificity of the current blockade for each nucleotide and to increase the dwell time in the construction zone. Both of these effects are likely to reduce the contribution of neighboring nucleotides to the current blockade, which is currently 4-5 nucleotides (Manrao et al., PloS One 6(10):e25723), and concomitantly reduce the high raw data error rates in nanopore sequencing (Dohm et al., NAR Genom Bioinform. 2(2):lgaa037 (2020)). (ii) Single-chain MspA can be used to slightly alter the diameter of the pore to modulate the interactions between amino acids in the constriction zone and nucleotides. This can be achieved by changing the subunit stoichiometry as shown herein (FIG. 2). In a simple geometrical model adding or removing a subunit alters the diameter of the MspA pore by ⅛^{th}, which is equivalent to approximately 1.5 Å, a reasonable range for significantly changing chemical interactions. Both the hexameric and heptameric pores are functional demonstrating that this approach is feasible. (iii) Motor proteins such as DNA polymerases employed to control the translocation rate of DNA through the pore have been instrumental for sequencing (Manrao et al., Nat Biotechnol. 30(4):349-35 (2012)) and have become standard in nanopore sequencing devices (Jain et al., Genome Biol. 17(1):239 (2016). However, the long distance between the motor proteins and the MspA constriction zone descreases the positional precision and increases error due to thermal motions of the flexible DNA strand (Lu et al., Biophys. J. 109(7): 1439-1445 (2015)) and the motor protein. Random positioning of the motor protein on top of the MspA pore might enhance this error. A single cysteine in one of the surface loops of single-chain MspA would enable the covalent attachment of the motor protein, eliminating the random positioning of motor and assembly with the MspA pore, and thereby reducing the system variability. (iv) It is likely that a specific path for single-stranded DNA inside of the MspA pore can be created, thereby decreasing the translocation velocity and eliminating the need for a motor protein altogether.

### Heterogeneous Channel Conductances of MspA Pores

The studies described herein showed a wide variety of channel conductances for single-chain MspA constructs ranging from 0.5 nS to 4.5 nS for sc₈MspA M2 without tags (FIG. 8D). This heterogeneity makes identification of single-chain MspA pores suitable for sequencing more difficult. However, this is not a consequence of the covalent linkers between the subunits of single-chain MspA as the channel conductances of the unmodified octameric MspA pore isolated from M. smegmatis also vary between 2 and 5 nS (FIG. 8A). These measurements are consistent with early measurements indicating that the conductance variability is an intrinsic property of the MspA pore. The mutations introduced in MspA M1 and MspA M2 to enable DNA translocation (Butler et al., 2008) appear to increase the intrinsic conductance variability but shift the major conductance peak from 4.5-5 nS to app. 1.5 nS (FIGS. 8B, C), indicating a significant contribution of the constriction zone to the overall channel conductance of MspA. It is plausible that neutralizing the constriction zone by mutating the negatively charged aspartates D90 and D91 to asparagines leads to a smaller and more flexible constriction zone due to the lack of electrostatic repulsion explaining both the shift to smaller channel conductances and their wider range. Harsh procedures used to extract MspA from M. smegmatis such as organic solvents (Niederweis, 1999) or boiling in detergents (Heinz et al., Anal. Biochem. 285(1): 113-120 (2000)) may contribute to pore heterogeneity, but it is striking that the recombinant single-chain MspA M2 pores, which were isolated from E. coli, showed a similar conductance profile as octameric MspA M2 isolated from M. smegmatis. Thus, it appears that pore conductance heterogeneity is largely an intrinsic property of MspA, driven mainly by the properties of the constriction zone. The hypothesis that the flexible constriction zone of the MspA variants M1 and M2 used in DNA sequencing experiments and in the corresponding single-chain variant sc₈MspA M2 is major driver of the conductance heterogeneity of these pores is supported by an additional experimental observation. The MspA PN1 variant with a phenylalanine 97 at the tip of the loop 6, which connects the constriction zone with the subsequent β-strand has a much smaller conductance distribution (FIG. 9A), indicating that putative hydrophobic interactions of neighboring phenylalanines stabilize the constriction zone. Conductance heterogeneity is common in general diffusion pores from other bacteria. For example, OmpF, the main porin of E. coli, and porins of Salmonella and Pseudomonas aeruginosa show a broad range of channel conductances. By contrast, the channel conductances are much better defined for specific porins, which have specific substrate binding sites, and the much smaller ion channels, which are located in the cytoplasmic membrane. For example, LamB, a maltodextrine-specific porin of E. coli (Nakae et al., J. Bacteriol. 142(3): 735-740 (1979); Klebba et al., Res. Microbiol. 153(7): 417-424 (2002); Hancock et al., J. Bacteriol. 174(2): 471-476 (1992)), OprO, a phosphate-specific porin of P. aeruginosa (Hancock et al., 1992) and the bacterial amyloid secretion channel CsgG (Goyal et al., Nature 516 (7530): 250-253) have narrow conductance distributions.

Single-Chain MspA Pores with Different Subunits Stoichiometries.

scMspA pores were constructed with different subunit stoichiometries to demonstrate the feasibility of changing the channel diameter, an important feat for nanopore sensing applications. All four single-chain MspA constructs with less than eight subunits formed functional pore proteins despite the likely limited tolerance of the MspA structure, in particular of the β-barrel, for expansions or reductions of the number of subunits. This is probably due to the self-assembly of the scMspA constructs with three and five subunits considering that the efficiency of the assembly of covalently linked subunits to a functional pore probably decreases with larger deviations from the octameric pore, which was found previously to be the most dominant form in a self-assembly process with the purified MspA monomer. Interestingly, the sc₈MspA PN1 pore had a much broader channel distribution than octameric MspA PN1 indicating that the purification and refolding procedure of recombinant scMspA introduced channel heterogeneity, which is not observed for octameric MspA PN1 purified from M. smegmatis. Thus, sc₃MspA PN1 assembles mostly to a hexameric structure. This is consistent with the conductance profile obtained for sc₃MspA PN1, which resembles that of sc₆MspA PN1 (FIG. 9B, D). The few additional larger channels for sc₃MspA PN1 might have been from a nonameric MspA pore. The conductance distribution of sc₈MspA PN1 is shifted to larger values compared to sc₈MspA PN1 indicating the presence of mainly decameric MspA pores. The conductance profile of sc₆MspA PN1 is similar to that of sc₈MspA PN1, while the conductances of sc₇MspA are shifted to larger values indicating assembly to larger pores (FIG. 9). As shown herein, single-chain MspA pores with varied stoichiometry can be produced and used in DNA sequencing. The unique adaptability of the single-chain MspA pore as a biosensor by making asymmetric mutations has enormous potential to improve the DNA sequencing capability of MspA and for many other applications such as detection of RNA, proteins and small molecules. The single-chain concept could also be applicable to other oligomeric protein pore, for example, CsgG (Cao et al., PNAS USA 111(50):E5439-5444 (2014)), ClyA (Mueller et al., Nature 459(7247):726-730 (2009)), FraC (Tanaka et al., Nat. Commun. 6:6337 (2015)) and α-HL (Song et al., Science 274(5294): 1859-18666 (1996)).

### Additional Single-Chain MspA Studies

As described above, single-chain MspA is capable of translocating DNA and has the same DNA recognition abilities as MspA produced from monomers. The rate of insertion into membranes with a small diameter (for example, 100 μm) was lower when compared to MspA M2 produced from monomers. Electron microscopy of negatively stained scMspA sample showed that scMspA formed spherical aggregates (FIG. 1A) which was consistent with previous reports. These aggregates likely formed predominantly between the linkers of scMspA molecules due to hydrophobic interactions. To reduce unspecific interactions and prevent aggregation, positive charges were introduced in the linker regions of scMspA (Table 6).

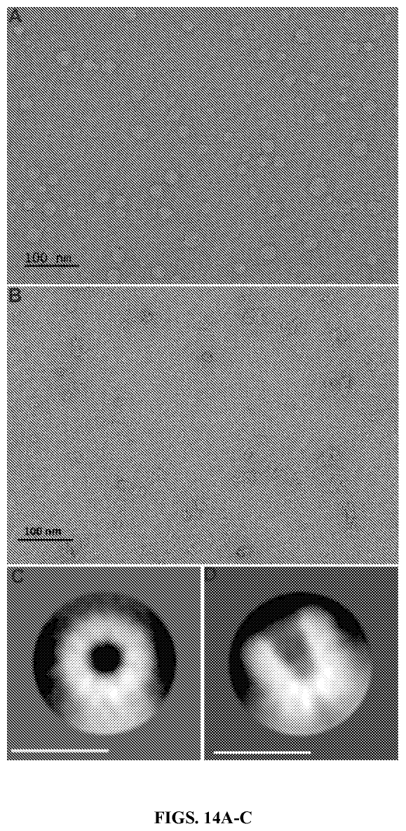

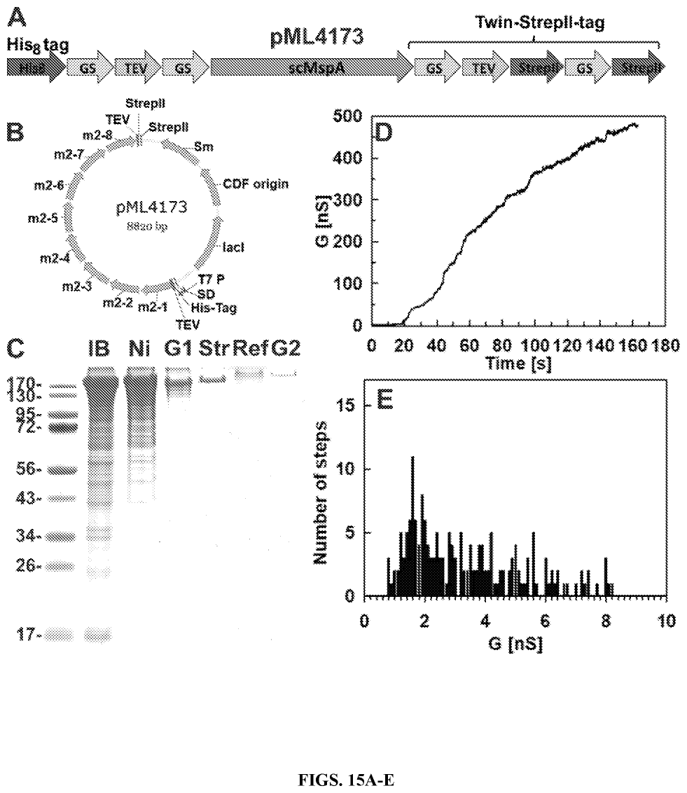

These linkers are referred to as ‘acidic linkers’. Similar to pML4170, the length of the linker was kept at 19 amino acids, some of the glycine residues were replaced with aspartic acid residues thus increasing negative charge of the linkers. Plasmid p-L4173, carrying sc8MspA M2, where all eight subunits have the same mutations (D90N/D91N/D93N/D118R/D134R/E139K) and are connected with acidic linkers, was constructed. The protein had a His8-tag on the N-terminus, and a Twin-StrepII tag on the C-terminus (FIG. 15 A, B). To purify full-length sc₈MspA, the protein was produced in E. coli and purified by following steps: 1) Ni(II)-affinity, 2) size exclusion chromatography, 3) Strep-tag affinity, 4) refolding in OPOE buffer, and 5) size exclusion chromatography. The NaCl concentration in the refolding buffer was increased to 300 mM to reduce sc₈MspA M2 protein aggregation during this step. The reformulated refolding buffer comprised 300 mM NaCl, 50 mM NaH2PO4/Na2HPO4, 200 mM L-Arginine, 800 mM urea, 0.5% n-octyl-POE (OPOE), pH 8.0. This purification scheme yielded 400 μg of apparently pure sc8MspA M2 protein per L of E. coli culture (FIG. 15 C). To inspect this preparation for the presence of aggregates, the protein was stained with 1% uranyl formate and the sample was analyzed under an electron microscope (FIG. 14 B, C, D). Large spherical aggregates were no observed in this preparation (FIG. 14B). 2D class averaging analysis of 2895 negatively stained particles showed that sc8MspA M2 from pML4173 had a central channel (approximately 10 nm outer diameter and 3 nm inner diameter) (FIG. 14C), and a distinctive cone-like geometry (FIG. 14D). These images are in a good agreement with MspA crystal structure (3, 4) and demonstrate the efficacy of this improved construct and purification method. To examine whether sc8MspA M2 formed functional channels lipid bilayer experiments were perfomred in a Montal-Mueller setup in DPhPC lipid bilayers using cuvettes with an aperture diameter of 1 mm. Addition of protein sample after final size exclusion chromatography resulted in a step-wise increase in current indicative of scMspA insertion into lipid bilayers (FIG. 15 D). It should be noted that very fast protein insertion was observed where the first, single insertion was evident within 20-30 seconds after protein addition to the cuvette (FIG. 15 D). In previous preparations, first insertion into lipid bilayer was observed, on average, between 200 to 400 seconds. Also, the protein concentration in the cuvette was 12 ng/ml which is less than 50-250 ng/ml when compared to the previous protein preparations from pML4170. Analysis of 181 opening events obtained from 3 membranes showed broad distribution histogram (FIG. 15 E) consistent with previous results. The predominant peak was at 1.6 nS. Overall, in this exemplary study, sc8MspA M2 with acidic linkers is less prone to aggregation when about 300 mM NaCl concentration was used during refolding.

TABLE 6
Linkers
Negatively charge aspartates which were introduced are in bold.

| Linkers | | | Modified | | Net |
|---|---|---|---|---|---|
| (original) | | Net | Acidic | | Charge |
| in | | Charge | linkers in | | at |
| pML4170 | Sequence | pH 7.0 | pML4173 | Sequence | pH 7.0 |
| 1 | GTGGGGSGGGGSGGGG | 0.1 | 1A | GTDGGGDGGGGDGGGGD | −3.9 |
| | SMH (SEQ ID NO: 23) | | | MH (SEQ ID NO: 53) | |
| 2 | STGGGGSGGGGSGGGG | 0 | 2A | STDGGGDGGGGDGGGGDA | −4 |
| | SASV (SEQ ID NO: 25) | | | S (SEQ ID NO: 54) | |
| 3 | VNGGGGSGGGGSGGGG | 1 | 3A | VNDGGGDGGGGDGGGGD | −4 |
| | SSR (SEQ ID NO: 27) | | | GG (SEQ ID NO: 55) | |
| 4 | HMGGGGSGGGGSGGGG | −0.9 | 4A | HMDGGGDGGGGDGGGGD | −4.9 |
| | SDI (SEQ ID NO: 29) | | | DI (SEQ ID NO: 56) | |
| 5 | LQGGGGSGGGGSGGGG | −1 | 5A | LQDGGGDGGGGDGGGGDF | −5 |
| | SFE (SEQ ID NO: 31) | | | E (SEQ ID NO: 57) | |
| 6 | GSGGGGSGGGGSGGGG | 1 | 6A | GSDGGGDGGGGDGGGGD | −4 |
| | STR (SEQ ID NO: 33) | | | GG (SEQ ID NO: 58) | |
| 7 | QLGGGGSGGGGSGGGG | 1 | 7A | QLDGGGDGGGGDGGGGD | −4 |
| | SLK (SEQ ID NO: 35) | | | GG (SEQ ID NO: 59) | |

Amino acid residues and their corresponding restriction sites:
GT-KpnI; MH-NsiI; ST-ScaI; AS-NheI; VN-HpaI; HM-NdeI; DI-EcoRV; LQ-PstI; FE-BstBI;
GS-BamHI; QL-PvulI

TABLE 7

| | | Net Charge |
|---|---|---|
| Linker | Sequence | at pH 7.0 |
| 1Ab | GTGGGGDGGGGDGGGGSGG | −2 |
| | (SEQ ID NO: 60) | |
| 2Ab | ASGGGGDGGGGDGGGGSGG | −2 |
| | (SEQ ID NO: 61) | |
| 3Ab | VNGGGGDGGGGDGGGGSGG | −2 |
| | (SEQ ID NO: 62) | |
| 4Ab | HMGGGGDGGGGDGGGGSGGI | −1.9 |
| | (SEQ ID NO: 63) | |
| 5Ab | LQGGGGDGGGGDGGGGSGG | −2 |
| | (SEQ ID NO: 64) | |
| 6Ab | GSGGGGDGGGGDGGGGSGG | −2 |
| | (SEQ ID NO: 65) | |
| 7Ab | QLGGGGDGGGGDGGGGSGG | −2 |
| | (SEQ ID NO: 66) | |

Amino acid residues and their corresponding
restriction sites:
GT-KpnI; MH-NsiI; ST-ScaI; AS-NheI; VN-HpaI;
HM-NdeI; DI-EcoRV; LQ-PstI; FE-BstBI; GS-BamHI;
QL-PvulI.

Msp Amino Acid Sequences

SEQ ID NO: 1
GLDNELSLVDGQDRTLTVQQWDTFLNGVFPLDRNRLTREWFHSGRAKYI
VAGPGADEFEGTLELGYQIGFPWSLGVGINFSYTTPNILIDDGDITAPP
FGLNSVITPNLFPGVSISADLGNGPGIQEVATFSVDVSGAEGGVAVSNA
HGTVTGAAGGVLLRPFARLIASTGDSVTTYGEPWNMN
SEQ ID NO: 2
GLDNELSLVDGQDRTLTVQQWDTFLNGVFPLDRNRLTREWFHSGRAKYI
VAGPGADEFEGTLELGYQIGFPWSLGVGINFSYTTPNILIDDGDITAPP
FGLNSVITPNLFPGVSISADLGNGPGIQEVATFSVDVSGPAGGVAVSNA
HGTVTGAAGGVLLRPFARLIASTGDSVTTYGEPWNMN
SEQ ID NO: 3
GLDNELSLVDGQDRTLTVQQWDTFLNGVFPLDRNRLTREWFHSGRAKYI
VAGPGADEFEGTLELGYQIGFPWSLGVGINFSYTTPNILIDDGDITGPP
FGLESVITPNLFPGVSISADLGNGPGIQEVATFSVDVSGPAGGVAVSNA
HGTVTGAAGGVLLRPFARLIASTGDSVTTYGEPWNMN
SEQ ID NO: 4
VDGQGRTLTVQQAETFLNGVFPLDRNRLTREWFHSGRATYHVAGPGADE
FEGTLELGYQVGFPWSLGVGINFSYTTPNILIDGGDITQPPFGLDTIIT
PNLFPGVSISADLGNGPGIQEVATFSVDVKGAKGAVAVSNAHGTVTGAA
GGVLLRPFARLIASTGDSVTTYGEPWNMN
SEQ ID NO: 5
MKAISRVLIAMVAAIAALFTSTGTSHAGLDNELSLVDGQDRTLTVQQWD
TFLNGVFPLDRNRLTREWFHSGRAKYIVAGPGADEFEGTLELGYQIGFP
WSLGVGINFSYTTPNILIDDGDITAPPFGLNSVITPNLFPGVSISADLG
NGPGIQEVATFSVDVSGAEGGVAVSNAHGTVTGAAGGVLLRPFARLIAS
TGDSVTTYGEPW
SEQ ID NO: 6
MTAFKRVLIAMISALLAGTTGMFVSAGAAHAGLDNELSLVDGQDRTLTV
QQWDTFLNGVFPLDRNRLTREWFHSGRAKYIVAGPGADEFEGTLELGYQ
IGFPWSLGVGINFSYTTPNILIDDGDITAPPFGLNSVITPNLFPGVSIS
ADLGNGPGIQEVATFSVDVSGPAGGVAVSNAHGTVTGAAGGVLLRPFAR
LIASTGDSVTTYGEPWNMN
SEQ ID NO: 7
MKAISRVLIAMISALAAAVAGLFVSAGTSHAGLDNELSLVDGQDRTLTV
QQWDTFLNGVFPLDRNRLTREWFHSGRAKYIVAGPGADEFEGTLELGYQ
IGFPWSLGVGINFSYTTPNILIDDGDITGPPFGLESVITPNLFPGVSIS
ADLGNGPGIQEVATFSVDVSGPAGGVAVSNAHGTVTGAAGGVLLRPFAR
LIASTGDSVTTYGEPWNMN
SEQ ID NO: 8
MRYLVMMFALLVSVTLVSPRPANAVDNQLSVVDGQGRTLTVQQAETFLN
GVFPLDRNRLTREWFHSGRATYHVAGPGADEFEGTLELGYQVGFPWSLG
VGINFSYTTPNILIDGGDITQPPFGLDTIITPNLFPGVSISADLGNGPG
IQEVATFSVDVKGAKGAVAVSNAHGTVTGAAGGVLLRPFARLIASTGDS
VTTYGEPWNMN

1. A method for preparing a purified population of single chain Mycobacterium smegmatis porin A (MspAs) comprising:
(a) expressing in E. coli α polypeptide comprising a single chain MspA, wherein the polypeptide comprises, in the following order,
(i) a first affinity tag, wherein the affinity tag is a polyhistidine tag;
(ii) a first amino acid linker;
(iii) a single chain MspA, wherein the single chain MspA comprises at least a first MspA monomer sequence and a second MspA monomer sequence; wherein the first and second monomer sequence are linked by a second amino acid linker;
(iv) a third amino acid linker; and
(v) a second affinity tag;
(b) recovering inclusion bodies that express the single chain MspAs from the E. coli under denaturing conditions;
(c) using Ni-affinity chromatography to obtain one or more fractions comprising single chain MspAs from the inclusion bodies under denaturing conditions;
(d) optionally separating the single chain MspAs in the one or more fractions using size exclusion chromatography to obtain a desired fraction comprising MspAs;
(e) purifying the MspAs from the one or more fractions of step (c) or the desired fraction of step (d) using second affinity tag purification under denaturing conditions;
(f) refolding the purified MspAs of step (e) in a refolding buffer, wherein the refolding buffer comprises about 50 mM to about 200 mM L-Arginine, about 800 mM to about 1000 mM urea, about 0.5% to about 1.0% OPOE, wherein the buffer has a pH of about 7.5 to about 8.5;
(g) concentrating the refolded MspAs using size exclusion chromatography to obtain a purified population of single chain MspAs.

2. The method of claim 1 wherein the refolding buffer further comprises about 150 mM to about 500 mM NaCl and/or about 25 mM to about 50 mM inorganic phosphates.

3. (canceled)

4. The method of claim 1, wherein the polypeptide comprises
(a) a first MspA monomer sequence
(b) a second MspA monomer sequence; and
(b) a third, fourth, fifth, sixth, seventh, and eighth MspA monomer sequence or any subset thereof,
wherein the first, second, third, fourth, fifth, sixth, seventh and eighth MspA monomer sequence or any subset thereof are arranged consecutively and wherein the second amino acid linker is positioned between any two Msp monomer sequences.

5. The method of claim 1, wherein the second amino acid linker is positioned between every two Msp monomer sequences.

6. The method of claim 1, wherein the second amino acid linker is an acidic amino acid linker having a net charge of about −2.0 to about −5.0, at pH 7.0.

7. The method of claim 1, wherein the polypeptide further comprises a protease cleavage site positioned between the first amino acid linker and the single-chain MspA and/or a protease cleavage site positioned between the third amino acid linker and the second affinity tag.

8. The method of claim 1, wherein the polypeptide comprises one or more first affinity tags, optionally separated by the first amino acid linker.

9. The method of claim 1, wherein the polypeptide comprises one or more second affinity tags, optionally separated by the third amino acid linker.

10. (canceled)

11. (canceled)

12. The method of claim 1, wherein at least one of MspA monomer sequences is a mutant monomer sequence.

13. The method of claim 12, wherein the mutant monomer sequence comprises an amino acid sequence having at least 95% identity to SEQ ID NO: 1.

14. The method of claim 12, wherein the mutant monomer sequence comprises a D90N, a D91N, and a D93N mutation.

15. The method of claim 14, wherein the mutant monomer sequence further comprises a D118 mutation, a D134 mutation, and a E139 mutation.

16. The method of claim 15, wherein the mutant monomer sequence further comprises a P97F mutation.

17. The method claim 12, wherein the mutant monomer sequence comprises a D90N, a D91N, a D93N mutation, a P97F mutation, a D118 mutation, a D134 mutation, a E139 mutation.

18. The method of claim 1, wherein single chain MspA comprises at least three MspA monomers, at least five MspA monomers or at least seven MspA monomers.

19. (canceled)

20. (canceled)

21. A system comprising a plurality of MspAs produced by any one of the methods of claim 1, the MspAs have a vestibule and a constriction zone that define a tunnel, wherein the tunnel is positioned between a first conductive liquid medium and a second conductive liquid medium, wherein at least one conductive liquid medium comprises an analyte, and wherein the system is operative to detect the analyte, when the system is subjected to an electric field sufficient to translocate the analyte from one conductive liquid medium to the other.

22.-26. (canceled)

27. A method for detecting the presence of an analyte, comprising:
a) applying an electric field sufficient to translocate an analyte from a first conductive medium to a second conductive medium in liquid communication through one or more MspAs produced by the method of claim 1, wherein the mutant Msp comprises a vestibule and a constriction zone that define a tunnel; and
b) measuring an ion current, wherein a reduction in the ion current indicates the presence of the analyte in the first medium.

28. The method of claim 27, further comprising identifying the analyte by comparing the current pattern to a current pattern obtained using a known analyte.

29. The method of claim 28, wherein a reduction in the current defines a blockade in the current pattern, and wherein identifying the analyte comprises comparing one or more blockades in the current pattern to one or more blockades in a known current pattern obtained using a known analyte.

30. The method of claim 28, wherein the analyte is a nucleotide, a nucleic acid, an amino acid, a peptide, a protein, a polymer, a drug, an ion, a pollutant, a nanoscopic object, or a biological warfare agent.

31.-36. (canceled)

37. A polypeptide comprising a single chain MspA, wherein the polypeptide comprises, in the following order,
(i) a first affinity tag, wherein the affinity tag is a polyhistidine tag;
(ii) a first amino acid linker;
(iii) a single chain MspA, wherein the single chain MspA comprises at least a first MspA monomer sequence and a second MspA monomer sequence; wherein the first and second monomer sequence are linked by a second amino acid linker;
(iv) a third amino acid linker; and
(v) a second affinity tag;

38. The polypeptide of claim 37, wherein the amino acid linker is positioned between every two Msp monomer sequences.

39. The polypeptide of claim 38 wherein the second amino acid linker is an acidic amino acid linker having a net charge of about −2.0 to about −5.0, at pH 7.0.

40. The polypeptide of claim 39, wherein the second amino acid linker is selected from the group consisting of SEQ ID NO: 53-SEQ ID NO: 59.

41. The polypeptide of claim 37, wherein each MspA monomer sequence has at least 95% identity to SEQ ID NO: 1.

42.-56. (canceled)


SEQUENCE LISTING (66 sequences)

SEQ ID NO: 1 - protein, 184 aa, Synthetic construct
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPWNMN

SEQ ID NO: 2 - protein, 184 aa, Synthetic construct
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 3 - protein, 184 aa, Synthetic construct
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 4 - protein, 176 aa, Synthetic construct
VDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 5 - protein, 208 aa, Synthetic construct
MKAISRVLIAMVAAIAALFTSTGTSHAGLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPW

SEQ ID NO: 6 - protein, 215 aa, Synthetic construct
MTAFKRVLIAMISALLAGTTGMFVSAGAAHAGLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 215 aa, Synthetic construct
MKAISRVLIAMISALAAAVAGLFVSAGTSHAGLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 8 - protein, 207 aa, Synthetic construct
MRYLVMMFALLVSVTLVSPRPANAVDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 9 - DNA, 12 bp, Synthetic construct
  source 1..12: mol_type = other DNA
gggsgggsggsa

SEQ ID NO: 10 - DNA, 20 bp, Synthetic construct
  source 1..20: mol_type = other DNA
gggsaggsasggsagggssa

SEQ ID NO: 11 - protein, 8 aa, Synthetic construct
HHHHHHHH

SEQ ID NO: 12 - protein, 18 aa, Synthetic construct
MHHHHHHHHENLYFQGEL

SEQ ID NO: 13 - protein, 29 aa, Synthetic construct
MHHHHHHHHGGGSGGGSGGSAENLYFQEL

SEQ ID NO: 14 - protein, 50 aa, Synthetic construct
MHHHHHHHHGGGSGGGSGGSAENLYFQGGGSAGGSASGGSAGGGSSAGEL

SEQ ID NO: 15 - skipped

SEQ ID NO: 16 - DNA, 10 bp, Synthetic construct
  source 1..10: mol_type = other DNA
ggsaggsasg

SEQ ID NO: 17 - protein, 8 aa, Synthetic construct
WSHPQFEK

SEQ ID NO: 18 - protein, 42 aa, Synthetic construct
GGGSGGSAENLYFQGWSHPQFEKSGGSAGGSASGWSHPQFEK

SEQ ID NO: 19 - skipped

SEQ ID NO: 20 - protein, 6 aa, Synthetic construct
ENLYFQ

SEQ ID NO: 21 - protein, 75 aa, Synthetic construct
AGKAGEGEIPAPLAGTVSKILVKEGDTVKAGQTVLVLEAMKMETEINAPTDGKVEKVLVKERDAVQGGQGLIKIG

SEQ ID NO: 22 - DNA, 15 bp, Synthetic construct
  source 1..15: mol_type = other DNA
ggggsggggsggggs

SEQ ID NO: 23 - protein, 19 aa, Synthetic construct
GTGGGGSGGGGSGGGGSMH

SEQ ID NO: 24 - DNA, 57 bp, Synthetic construct
  source 1..57: mol_type = other DNA
ggtaccggcggtggcggtagtggcggtggcggttccggcggtggcggttcaatgcat

SEQ ID NO: 25 - protein, 19 aa, Synthetic construct
STGGGGSGGGGSGGGGSAS

SEQ ID NO: 26 - DNA, 57 bp, Synthetic construct
  source 1..57: mol_type = other DNA
agtactggcggtggcggttcgggcggtggcggtagcggcggtggcggtagcgctagc

SEQ ID NO: 27 - protein, 19 aa, Synthetic construct
VNGGGGSGGGGSGGGGSSR

SEQ ID NO: 28 - DNA, 57 bp, Synthetic construct
  source 1..57: mol_type = other DNA
gttaacggcggtggcggttctggcggtggcggtagtggcggtggcggtagctctaga

SEQ ID NO: 29 - protein, 19 aa, Synthetic construct
HMGGGGSGGGGSGGGGSDI

SEQ ID NO: 30 - DNA, 57 bp, Synthetic construct
  source 1..57: mol_type = other DNA
catatgggcggtggcggttccggcggtggcggttcaggcggtggcggttcggatatc

SEQ ID NO: 31 - protein, 19 aa, Synthetic construct
LQGGGGSGGGGSGGGGSFE

SEQ ID NO: 32 - DNA, 57 bp, Synthetic construct
  source 1..57: mol_type = other DNA
ctgcagggcggtggcggtagcggcggtggcggttctggcggtggcggtagtttcgaa

SEQ ID NO: 33 - protein, 19 aa, Synthetic construct
GSGGGGSGGGGSGGGGSTR

SEQ ID NO: 34 - DNA, 57 bp, Synthetic construct
  source 1..57: mol_type = other DNA
ggatccggcggtggcggttcaggcggtggcggttcgggcggtggcggtagcacgcgt

SEQ ID NO: 35 - protein, 19 aa, Synthetic construct
QLGGGGSGGGGSGGGGSLK

SEQ ID NO: 36 - DNA, 57 bp, Synthetic construct
  source 1..57: mol_type = other DNA
cagctgggcggtggcggttctggcggtggcggtagtggcggtggcggttcccttaag

SEQ ID NO: 37 - DNA, 20 bp, Synthetic construct
  source 1..20: mol_type = other DNA
cgttctcggctcgatgatcc

SEQ ID NO: 38 - DNA, 23 bp, Synthetic construct
  source 1..23: mol_type = other DNA
gcgagtcagtgagcgaggaagcg

SEQ ID NO: 39 - DNA, 27 bp, Synthetic construct
  source 1..27: mol_type = other DNA
ggtaacatcaccgctttcccgttcggc

SEQ ID NO: 40 - DNA, 15 bp, Synthetic construct
  source 1..15: mol_type = other DNA
tgaatggatccggcg

SEQ ID NO: 41 - DNA, 27 bp, Synthetic construct
  source 1..27: mol_type = other DNA
ttttttaagcttgtcacccagctggtt

SEQ ID NO: 42 - DNA, 15 bp, Synthetic construct
  source 1..15: mol_type = other DNA
gctcgctgcagggcc

SEQ ID NO: 43 - DNA, 27 bp, Synthetic construct
  source 1..27: mol_type = other DNA
ttttttaagctttcagccggatccatt

SEQ ID NO: 44 - DNA, 15 bp, Synthetic construct
  source 1..15: mol_type = other DNA
aaccatatgggcggt

SEQ ID NO: 45 - DNA, 27 bp, Synthetic construct
  source 1..27: mol_type = other DNA
ttttttaagctttcagccctgcaggtt

SEQ ID NO: 46 - DNA, 15 bp, Synthetic construct
  source 1..15: mol_type = other DNA
tgaacagtactggcg

SEQ ID NO: 47 - DNA, 28 bp, Synthetic construct
  source 1..28: mol_type = other DNA
ttttttaagctttcagccgttaacgttc

SEQ ID NO: 48 - DNA, 15 bp, Synthetic construct
  source 1..15: mol_type = other DNA
tgaacagtactggcg

SEQ ID NO: 49 - DNA, 84 bp, Synthetic construct
  source 1..84: mol_type = other DNA
gctggctctgttgctctctcgcaacagagccagctttttttttttttttttttttttttttttttttttttttttttttttttt

SEQ ID NO: 50 - DNA, 84 bp, Synthetic construct
  source 1..84: mol_type = other DNA
gctggctctgttgctctctcgcaacagagccagcaaaaaaaaaaaaaaaaaaaaaaaaaaaaaaaaaaaaaaaaaaaaaaaaaa

SEQ ID NO: 51 - DNA, 60 bp, Synthetic construct
  source 1..60: mol_type = other DNA
gctggctctgttgctctctcgcaacagagccagccccccccccccccccccccccccccc

SEQ ID NO: 52 - DNA, 84 bp, Synthetic construct
  source 1..84: mol_type = other DNA
gctggctctgttgctctctcgcaacagagccagcgggaaaaaaaaaaaaaaaaaaaaaaaaaaaaaaaaaaaaaaaaaaaaaaa

SEQ ID NO: 53 - protein, 19 aa, Synthetic construct
GTDGGGDGGGGDGGGGDMH

SEQ ID NO: 54 - protein, 19 aa, Synthetic construct
STDGGGDGGGGDGGGGDAS

SEQ ID NO: 55 - protein, 19 aa, Synthetic construct
VNDGGGDGGGGDGGGGDGG

SEQ ID NO: 56 - protein, 19 aa, Synthetic construct
HMDGGGDGGGGDGGGGDDI

SEQ ID NO: 57 - protein, 19 aa, Synthetic construct
LQDGGGDGGGGDGGGGDFE

SEQ ID NO: 58 - protein, 19 aa, Synthetic construct
GSDGGGDGGGGDGGGGDGG

SEQ ID NO: 59 - protein, 19 aa, Synthetic construct
QLDGGGDGGGGDGGGGDGG

SEQ ID NO: 60 - protein, 19 aa, Synthetic construct
GTGGGGDGGGGDGGGGSGG

SEQ ID NO: 61 - protein, 19 aa, Synthetic construct
ASGGGGDGGGGDGGGGSGG

SEQ ID NO: 62 - protein, 19 aa, Synthetic construct
VNGGGGDGGGGDGGGGSGG

SEQ ID NO: 63 - protein, 20 aa, Synthetic construct
HMGGGGDGGGGDGGGGSGGI

SEQ ID NO: 64 - protein, 19 aa, Synthetic construct
LQGGGGDGGGGDGGGGSGG

SEQ ID NO: 65 - protein, 19 aa, Synthetic construct
GSGGGGDGGGGDGGGGSGG

SEQ ID NO: 66 - protein, 19 aa, Synthetic construct
QLGGGGDGGGGDGGGGSGG